(19) United States
(12) Patent Application Publication
(10) Pub. No.: US 2014/0076404 A1
(43) Pub. Date: Mar. 20, 2014

(54) IR-ACTIVATED PHOTOELECTRIC SYSTEMS

(71) Applicants: Mei-Chee Tan, Piscataway, NJ (US); Richard E Riman, Belle Mead, NJ (US)

(72) Inventors: Mei-Chee Tan, Piscataway, NJ (US); Richard E Riman, Belle Mead, NJ (US)

(21) Appl. No.: 13/994,861

(22) PCT Filed: Dec. 15, 2010
(86) PCT No.: PCT/US10/60513

§ 371 Date: Dec. 2, 2013

(51) Int. Cl. H01L 31/0264; A61L 9/18; B08B 7/00; H01G 9/20

(52) U.S. Cl. CPC H01L 31/0264; H01G 9/2031; A61L 9/18; B08B 7/0035
USPC 136/263; 204/158.2; 204/157.5; 204/157.3; 204/157.52; 204/157.6; 204/157.9; 252/501.1; 422/22; 134/1

(57) ABSTRACT

Photoelectric systems combining a semiconductor and a phosphorescent compound with an emission spectrum of photons with energy levels equal to or greater than the activation energy of the semiconductor, wherein the phosphorescent compound is characterized by the emission spec-tram being produced by excitation of the phosphorescent compound with lower energy photons and the separation distance between the semiconductor and the phosphorescent compound is less than the distance at or above which scattering losses predominate. Methods are that embody technological applications of the photoelectric systems are also disclosed, as well as articles that embody technological applications of the photoelectric systems.

### TECHNICAL FIELD

The present invention relates to photoelectric systems in which semiconductors that are activated by ultra-violet wavelength (UV) photons, including semiconductors that are activated by both UV and visible wavelength photons, are combined with up-converting phosphors that emit UV photons upon excitation with infrared wavelength (IR) photons, including phosphors that emit both UV and visible wavelength photons upon excitation with IR photons, so that exposure of the combination to IR radiation activates the semiconductor to generate a photo-catalytic or photovoltaic effect. The present invention also relates to photocatalytic and photo-voltaic methods, and devices employing the methods. The methods and devices include, but are not limited to, methods and devices that purify air and water, remediate chemical wastes, generate electricity, treat cancer, produce hydrogen fuel from water, clean and sterilize objects and surfaces, and the like.

### BACKGROUND ART

Photoelectric devices generate charge carriers in the form of electrons and holes upon the device's exposure of light. The photoelectric effect is a phenomenon where light falling on matter, typically a semiconductor, generates charge carriers (i.e. electrons and holes). Exposure to photons of appropriate wavelengths creates electron-hole pairs in the semiconductor that react with any surface or adsorbed water, water vapor, oxygen, carbon dioxide or organic materials to generate free radicals and other reactive species. Photoelectric methods and devices embody technological applications of photocatalytic and photovoltaic systems.

Photoelectric devices convert light to either electrical or chemical (redox) energy as a result of light acting as an electron pump. Absorption of a photon of light by an atom or molecule pumps an electron from a lower energy state to a higher one, which results in the formation of an electron-hole pair. The wavelength of light that causes such a transition is that with energy equal to or greater than the difference in energies of the two energy states, E_{g}. To utilize the light, separation of the electron-hole pair must be achieved to prevent undesired non-radiative electron-hole recombination. This separation can be initiated by an electric field (i.e. difference in electrical potential) or a “chemical field” (i.e. difference in chemical potential). If the electron-hole pair is separated so that the electron flows to a suitable acceptor species, or an electron from a suitable donor fills the photogenerated hole, then the light energy has been stored as chemical (redox) energy. If the electron is pumped through a wire, it will be converted to an electrical current flow.

Compared to other potential materials (e.g. CeO₂, ZnO, ZnS, CdS), TiO₂ is the most widely investigated semiconductor because of its chemical and biological inert nature, photo-catalytic stability and low environment risks. Photon absorption occurs when incident photon energy is at least equal to that of the TiO₂ bandgap, leading to the promotion of an electron from the valence band to the conduction band of TiO₂, and resulting in the generation of a hole in the valence band. Because the bandgap for anatase and rutile TiO₂ is 3.2 and 3.0 eV, which corresponds to wavelengths of 385 and 410 nm, respectively, ultraviolet light (λ≦380 nm) serves as an excitation source.

Anatase TiO₂ has been found to be the more active of the two phases for most photo-chemical and photovoltaic reactions. The photo-induced electron-hole pairs will either recombine or participate in chemical reactions with surface or adsorbed species. For example, the oxidation of water or hydroxide ion by the valence band hole can produce the hydroxyl radical (.OH). The conduction band electron can react with molecular oxygen to form the superoxide radical-anion, which can be involved in further reactions. In addition, the valence band hole and conduction band electron can also react directly with adsorbed pollutants. Current limitations to widespread industrial use of photoelectric systems are low photochemical and photovoltaic efficiency of semiconductors and scale-up problems.

The quantum yield for a photochemical or photovoltaic reaction, φ, can be used as a measure of photoelectric efficiency and is expressed as:

$$φ = \frac{rate of reaction induced by photon absorption}{flux of absorbed photons}$$

One of the approaches for increasing quantum yield of photocatalysts is to increase reaction rates by reducing electron-hole recombination through introduction of surface and volume defects. Such defects can be created by selective metal ion doping and tailoring TiO₂ particle sizes for various photochemical reactions. Alternatively, TiO₂ can be coated with organic dyes to “sensitize” and improve photon absorption leading to increased electron injection and reaction rates.

Besides increasing reaction rates, efforts have also been made to improve the design and configuration of photoelectric reactors in order to reduce light transfer and mass transfer limitations. High intensity UV light sources (e.g. 300-950 W Xe lamps or 450 W Hg lamps) are typically required for the activation of photoelectric systems, because of the light transfer limitations inherent to UV. Most UV is lost through scattering wherein, according to Rayleigh scattering theory, the transmitted intensity, I is related to wavelength, λ, according to I∝λ⁻⁴.

Furthermore, because of the low availability of UV from the solar spectrum of ˜3% (Table 1), current photoelectric systems have not been able to effectively utilize energy from the sun. Current attempts to improve the utilization of light from the solar spectrum have mostly been focused on developing visible light sensitive TiO₂ photoelectric systems by anion doping (e.g. N, C) of TiO₂.

TABLE 1
Energy distribution in the terrestrial solar spectrum
(Air Mass, AM 1.5).

| Spectral | Wavelength | Energy | Contribution to |
|---|---|---|---|
| Region | (nm) | (eV) | Total Spectrum (%) |
| near-UV | 315-400 | 3.92-3.09 | 2.9 |
| Blue | 400-510 | 3.09-2.42 | 14.6 |
| Green/yellow | 510-610 | 2.42-2.03 | 16.0 |
| Red | 610-700 | 2.03-1.77 | 13.8 |
| near-IR | 700-920 | 1.77-1.34 | 23.5 |
| Infrared | 920->1400 | 1.34-<0.88 | 29.4 |

Photoelectric methods and devices offer a low-temperature, non-energy intensive approach for chemical waste remediation, self-cleaning applications, microorganism sterilization, aseptic processing, water and air purification, energy generation and medical treatment. There remains a need for means by which photoelectric methods and devices can more effectively utilize the solar spectrum.

### SUMMARY OF THE INVENTION

The present invention addresses these needs by providing a photoelectric system that utilizes solar energy more efficiently by exploiting the IR portion of the solar spectrum, which, as shown in Table 1, is approximately 7 to 10 times more available than UV. Therefore, accord-ing to one aspect of the present invention, a photoelectric system is provided combining a semi-conductor with a phosphorescent compound capable of emitting photons with energy levels equal to or greater than the activation energy of the semiconductor upon excitation with lower energy photons, wherein the separation distance between the semiconductor and phosphorescent compound is less than the distance at or above which scattering losses dominate.

The maximum separation distance will depend on the optical properties of the medium in which system will be immersed. The microstructure and hierarchy of the photoelectric system is engineered according to the reaction or application of interest, by controlling variables like composite composition and particle size.

One embodiment of this aspect of the invention provides a photoelectric system in which the phosphorescent compound is an upconverting phosphor that emits photons with energy levels equal to or greater than the activation energy of the semiconductor upon excitation with photons with an energy level of about 2.0 eV or less. In a more specific embodiment, the upconverting phosphor is excited by IR wavelength photons.

In a more specific embodiment, the upconverting phosphors are host compounds doped with rare earth elements. Suitable host compounds, rare earth dopants and methods of making phosphor compounds are disclosed by U.S. Pat. Nos. 6,699,406 and 7,094,361, the disclosures of which are incorporated herein by reference.

The absorption and emission properties of rare earth doped phosphors can be tailored by controlling the local environment, such as site symmetry, crystal field strength and electron-phonon interaction strength of rare-earth dopants. Halide hosts (e.g. NaYF₄, YF₃, LaF₃) are favored for their low phonon energies which minimize non-radiative losses to enable intense up-converting emissions. While all rare earth elements are excited to some extent by IR-wavelength photons and emit to some extent UV-wavelength photons, phosphors doped with ytterbium (Yb) and one or more elements selected from thulium (Tm), erbium (Er) and gadolinium (Gd) are preferred. Specific embodiments of rare earth doped phosphors suitable for use with the present invention include NaYF₄:Yb—Tm, NaGdF₄:Yb—Tm LaF₃:Yb—Tm, YF₃:Yb—Tm, GdF₃:Yb—Tm, YF₃:Yb—Gd—Tm and NaYF₄:Yb—Er phosphors.

In another embodiment of this aspect of the present invention the semiconductor is anatase or rutile titanium dioxide that is activated by UV photons and the phosphorescent compound emits UV photons with energy levels equal to or greater than the activation energy of the titanium dioxide. According to another embodiment according to this aspect of the invention, the titanium dioxide is doped to reduce the semiconductor band gap energy to permit activation by photons with visible wavelength energy levels and the phosphorescent compound emits visible and UV wavelength photons with energy levels equal to or greater than the activation energy of the doped titanium dioxide.

Phosphorescent compounds and semiconductors at different length scales (nano-, micro- and macroscales) and forms (e.g. non-porous and/or porous) can be integrated together according to the various arrangements in FIG. 3. Each of the schemes shown in FIG. 3 can be dispersed or deposited in various matrices (e.g. air or water) and supports (e.g. stainless steel, glass, polymers), or function alone without a matrix or other support.

Therefore, in another embodiment of this aspect of the invention, the phosphorescent compounds and semiconductors are integrated in the form of a core-shell microstructure in which a continuous semiconductor shell layer covers a phosphorescent compound core. The core-shell microstructure is not limited only to particles with a spherical morphology, and can be further extended to apply to platelets, prisms, rods, fibers and cubes.

In another embodiment of this aspect of the invention, a mixture is provided of both phosphorescent compounds and semiconductors having either ordered or disordered arrangements. The phosphorescent compound and semiconductor particles can independently have either of the following morphologies: spheres, rods, tubes, prisms, platelets, fibers and cubes. That is, the morphologies can be the same or different. The mixture can be further compacted to form a solid pellet or tablet using conventional ceramic pressing technologies (e.g. hydraulic press and hot press). Alternatively, these mixtures can be dispersed in another external matrix (liquid or gas) or be supported on a porous solid matrix (e.g. zeolites or fiber networks).

In another embodiment of this aspect of the invention, a mesh is provided of an interpenetrating fiber network of phosphorescent compound and semiconductor fibers. The fibers can be porous or non-porous. The interpenetrating network of fibers may be applied to current engineering applications without further fabrication (e.g. 2-dimensional planar sheet of inter-penetrating fibers) immersed in a liquid (e.g. water) or gas (e.g. air), or supported on solids (e.g. spun together with cotton or nylon fibers). In a specific embodiment, fibers or tubes of phosphorescent compounds and semiconductors can be arranged to have an ordered configuration.

In yet another embodiment of this aspect of the invention, the phosphorescent compounds can be embedded within a continuous matrix of the semiconductor. The semiconductor matrix can be porous or non-porous and have various geometries and forms such as spheres, cubes, rods, tubes, prisms, films, sheets, and the like.

In another embodiment of this aspect of the invention a continuous layer of semiconductor can be coated onto a film or sheet of a phosphorescent compound to form a continuous bi-layer structure as shown in FIG. 3. Each layer of material can be porous or non-porous. A plurality of bi-layers can be assembled to provide the multilayer structure shown in FIG. 3

According to another aspect of the present invention, photoelectric methods and devices are provided that embody technological applications of the photoelectric systems of the present invention. Photoelectric devices generate charge carriers in the form of electrons and holes upon the device's exposure of light. The photoelectric effect is a phenomenon where light falling on matter generates charge carriers (i.e. electrons and holes).

Photoelectric devices, and methods implemented by the devices, therefore include photo-voltaic devices and methods and photocatalytic devices and methods. In photovoltaic devices and methods, photogenerated electrons and holes in various material structures are transported to external circuits (i.e. enable electricity generation). Photocatalytic devices and methods enable the conversion of light photons (e.g. solar energy) into chemical energy in situ by utilizing photogenerated electrons and holes for redox reactions.

For example, photocatalytic devices and methods embodying the photoelectric systems of the present invention enable the conversion of light photons (e.g. solar energy) into chemical energy in situ by utilizing photogenerated electrons and holes for redox reactions. Photocatalytic devices and methods can therefore be used for chemical waste remediation. According to one embodiment, a photocatalytic chemical waste remediation method is provided in which material contaminated with volatile organic species is purified by contacting material containing volatile organic species for remediation with the photoelectric system of the present invention and irradiating the photoelectric system with photons of sufficient energy to excite the phosphorescent compound to emit photons of sufficient energy to activate the semiconductor to generate species that degrade or decompose the volatile organic species.

Photocatalytic devices and methods are also provided in which the photoelectric systems of the present invention are fabricated as self-cleaning and self-sterilizing surfaces. In an embodiment, a method is provided for cleaning and sterilizing surfaces in which a surface coated with the photoelectric system of the present invention is irradiated with photons of sufficient energy to excite the phosphorescent compound to emit photons of sufficient energy to activate the photocatalyst to generate species that kill the microbes or degrade or decompose organic substances on the coated surface. This method is particularly well-suited for aseptic processing lines required in food processing and pharmaceutical plants to enable sterile processing and packaging.

In a specific embodiment, removal of fouling agents and bacterial films from implanted medical devices can be completed without the need of undergoing surgical procedures. Methods according to this embodiment implant a medical device coated with the photoelectric system of the present invention in which the phosphorescent compound is a rare earth doped upconverting phosphor that upon excitation with IR wavelength photons emits UV wavelength photons cap-able of activating the titanium dioxide semiconductor, and irradiating the medical device with IR light, so that the non-invasive, deep tissue penetrating IR light excites the upconverting phosphor to emit UV wavelength photons to activate the photocatalyst to generate species that degrade and remove any undesirable fouling protein or microbial populations that would otherwise impede the performance of the implanted medical devices.

In another specific embodiment, outside exterior surfaces exposed to the elements are cleaned and sterilized by a source of ambient light. Methods according to this embodiment coat the exterior surface with a photoelectric system according to the present invention in which the phosphorescent compound is a rare earth doped upconverting phosphor that upon excitation with IR wavelength photons emits UV wavelength photons capable of activating the semiconductor, so that exposure to ambient light sources containing IR wavelength photons excite the upconverting phosphor to emit UV wavelength photons to activate the semiconductor to generate species that degrade or decompose any contaminants on the coated surface. In a more specific embodiment, the source of ambient light containing IR wavelength photons is the sun.

Photocatalytic methods and devices embodying the photoelectric system of the present invention can also be used to generate ozone. According to this embodiment, methods for generating ozone are provided by contacting the photoelectric system of the present invention with an oxygen source and irradiating the photoelectric system with photons of sufficient energy to excite the phosphorescent compound to emit photons of sufficient energy to activate the semi-conductor and generate species that produce ozone from oxygen. The oxygen source may be atmospheric, i.e., unprocessed air, that is contacted with the photoelectric system of the present invention under ambient conditions.

Photocatalytic methods and devices embodying the photoelectric system of the present invention can also be used to purify contaminated air and water. According to this embodiment, methods are provided for purifying air or water contaminated with microbes or undesirable organic compounds or organic matter by contacting the photoelectric system of the present invention with an air or water source contaminated with microbes or undesirable organic com-pounds or organic matter and irradiating the photoelectric system with photons of sufficient energy to excite the phosphorescent compound to emit photons of sufficient energy to activate the semiconductor and generate species that purify the air or water by killing the microbes or degrade or decompose the undesirable organic compounds or organic matter.

In a specific embodiment, the phosphorescent compound is a rare earth doped upconverting phosphor that upon excitation with IR wavelength photons emits UV wavelength photons capable of activating the titanium dioxide semiconductor and the photoelectric system is irradiated with ambient light. In a more specific embodiment the source of ambient light is the sun. In another more specific embodiment, outdoor air is purified by coating buildings and other structures with the photoelectric system of the present invention using IR-excited phosphorescent compounds so that the coated buildings and structures purify the air upon exposure to sunlight.

Photocatalytic methods and devices embodying the photoelectric system of the present invention can also be used to generate fuels like hydrogen or methane. According to this embodiment, a method for the photoelectric production of hydrogen or a hydrocarbon fuel is provided in which the photoelectric system of the present invention is contacted with a source of hydrogen or a source of hydrocarbon fuel and irradiated with photons of sufficient energy to excite the phosphorescent compound to emit photons of sufficient energy to activate the semi-conductor and generate species that decompose the hydrogen source to produce hydrogen or the hydrocarbon fuel source to produce hydrocarbon fuel. The hydrogen source may be essentially any compound that can be photocatalytically decomposed to produce hydrogen, such as water, methanol, and the like. Hydrocarbon fuels such as methane, methanol and formaldehyde can be generated from sources like biomass (e.g. lignocelluloses) and carbon dioxide.

Carbon dioxide emissions from industrial and combustion processes are the largest contributor among greenhouse gases. Besides converting undesired carbon dioxide into more useful compounds (e.g. methanol, methane, etc.), this technology will enable a method of reducing carbon dioxide that is less energy-consuming compared to other conventional fuel generation methods.

Some of the factors that will affect the photocatalytic performance in fuel generation are wavelength of ultraviolet light, pressure, temperature, solvents (water, acetonitrile, isopropanol) and moisture content (i.e. carbon dioxide to water ratio). Using the IR-activated photocatalyst system of the present invention will improve the efficiency of solar powered fuel generation by reducing light transfer limitations.

In a specific embodiment the phosphorescent compound is a rare earth doped upconverting phosphor that upon excitation with IR wavelength photons emits UV wavelength photons capable of activating the titanium dioxide semiconductor and the photoelectric system is irradiated with ambient light. In a more specific embodiment, the source of ambient light is the sun, so that hydrogen is generated using the sun as the sole source of energy.

Photocatalytic methods and devices embodying the photoelectric system of the present invention can also be used in photodynamic cancer therapy. Methods according to this embodiment deliver to the site of a tumor in a patient the photoelectric system of the present invention in which the phosphorescent compound is a rare earth doped upconverting phosphor that upon excitation with IR wavelength photons emits UV wavelength photons capable of activating the titanium dioxide semiconductor, and irradiate the tumor with IR light, so that the non-invasive, deep tissue penetrating IR light excites the upconverting phosphor to emit UV wavelength photons to activate the semiconductor to generate species that kill tumor cells.

According to another aspect of the present invention, photocatalytic devices are provided that embody technological applications of the photoelectric systems of the present invention. For example, photoelectric systems according to the present invention can be easily integrated and adapted into existing chemical waste treatment plants. Fiber bundles of the IR-activated photo-electric system can be included within pipelines delivering the effluent waste streams to generate species that degrade or decompose organic species or organic matter within the waste streams.

The photoelectric systems can be applied as coatings on surfaces for architecture (e.g. windows, building facades), automotive (e.g. rear view minors), office (e.g. computer screens) and appliances (e.g. stove tops, refrigerators, television), thus imparting self-cleaning properties to these objects. The photoelectric systems of the present invention thus can be coated on articles to provide photocatalytic devices with self-cleaning surfaces. Naturally occurring fatty acids (e.g., octadecanoic (stearic) acid, hexadecanoic (palmitic) acid) can be photocatalytically degraded on the coatings, thus enabling removal of oily finger-prints and organic residues to make surfaces easier to clean. Besides removing organic residues, surface properties (e.g. hydrophilicity and hydrophobicity) can be controlled using the photoelectric systems of the invention. The photoelectric systems can also be coated on the surfaces of the hulls of ships and heat exchangers to prevent or reduce fouling (e.g. barnacles, algae, protein precipitates).

The photoelectric systems of the present invention can also be coated on the surface of articles to provide photocatalytic means for sterilizing the surface. The photoelectric systems can be coated onto various surfaces like cooking utensils, surgical tools, medical devices, biomedical implants, food packages and door knobs to allow easy and effective sterilization. Having door knobs and other frequently touched surfaces and objects (e.g. money, escalator handrails and elevator buttons) coated with IR-activated photoelectric systems will allow these surfaces and objects to remain sterile and subsequently prevent transmission of contagious diseases. Photoelectric systems according to the present invention can be integrated into aseptic processing lines required in food processing and pharmaceutical plants to enable sterile processing and packaging.

The photoelectric systems of the present invention can also be easily integrated and adapted into existing ozone generators, waste water treatment plants and water purification systems and devices for the purification of both indoor and outdoor air. The photoelectric system can be incorporated into current air filtration (e.g. on the filters) and circulation (e.g. on fans or surfaces of air vents) units and other HVAC system components found in office buildings, hospitals, vehicles (e.g. automobiles, trucks, army tanks, trains, airplanes), toilets and confined places to enable indoor air purification. For outdoor air purification, the photoelectric system can be incorporated into existing architectures (e.g. roof tiles), air systems in automobiles or vehicle exhaust systems. Having the photoelectric system incorporated on building facades and roofs instead of catalytic converters in automobiles will enable solar powered air purification.

The photoelectric systems of the present invention can be exploited in dye-sensitized solar cells to provide photovoltaic methods and devices for the efficient generation of electrical power. Dye-sensitized solar cells according to the present invention are provided in which the photoelectric system of the present invention is employed as the titanium dioxide layer. The photoelectric system of the present invention will improve the efficiency of solar powered energy generation by reducing light transfer limitations and enhancing electron injection rates by converting unused low energy photons to useful high energy photons.

When the phosphorescent compound is a rare earth doped upconverting phosphor that upon excitation with IR wavelength photons emits UV wavelength photons capable of activating the titanium dioxide semiconductor, IR radiation can be used instead of UV radiation to activate the photoelectric system. Besides low scattering losses and more efficient light transfer, another benefit of IR radiation is its deeper penetration depth in various systems (e.g. water, organic solvents and biological tissue). The deeper penetration will enable activation of semiconductors embedded deep within or supported by porous structures and matrices. UV light is localized to the vicinity of the semiconductor to enhance photoelectric performance. Energy transfer and UV emission is limited to the fine length scales of the microstructure of upconverting rare earth doped phosphors and TiO₂ and not the macroscopic length scales where UV emission could pose a safety hazard.

### BRIEF DESCRIPTION OF THE DRAWINGS

FIG. 1 (a) depicts the absorption spectrum of Degussa TiO₂ and FIG. 1 (b) depicts an XRD spectrum of Degussa TiO₂ (˜80-85% anatase);

FIG. 2 depicts photocatalytic reactions of TiO₂ in aqueous solutions;

FIG. 3 depicts microstructures and configurations of IR-activated photocatalyst systems according to aspects of the present invention;

FIG. 4 depicts integration of FIG. 3 microstructures and configurations into engineering applications;

FIG. 5 depicts IR upconversion of Gd³⁺, Yb³⁺ and Tm³⁺ co-doped systems, where ET and CR represents energy transfer and cross-relaxation, respectively;

FIG. 6 depicts the dissociation of methyl red in aqueous solutions;

FIG. 7 depicts absorption spectra of HMR and MR-ions;

FIG. 8 depicts XRD patterns of hexagonal as-synthesized NaY_{0.78}Yb_{0.20}Er_{0.02}F₄, NaY_{0.78}Yb_{0.20}Tm_{0.02}F₄ and NaY_{0.68}Yb_{0.20}Gd_{0.10}Tm_{0.02}F₄;

FIG. 9 (a) depicts IR-to-UV upconversion of NaY₀₇₈Yb_{0.20}Er_{0.02}F₄ and FIG. 9 (b) depicts IR-to-UV upconversion of NaY_{0.78}Yb_{0.20}Tm_{0.02}F₄;

FIG. 10 depicts IR-to-UV upconversion spectra of NaY_{0.68}Yb_{0.20}Gd_{0.10}Tm_{0.02}F₄ for varying pump powers;

FIGS. 11 (a)-(d) depict double logarithmic plots of the upconversion emission intensity with respect to excitation power for emission peaks of NaY_{0.68}Yb_{0.20} Gd_{0.10}Tm_{0.02}F₄ at (a) 335-361 nm, (b) 270-280 nm, (c) 311 nm and (d) 289 nm;

FIG. 12 depicts UV emission spectra of NaY_{0.78-x}Yb_{0.20}GdₓTm_{0.02}F₄ with varying Gd³⁺ dopant concentrations;

FIG. 13 depicts the integrated area of UV emissions from NaY_{0.78-x}Yb_{0.20}GdₓTm_{0.02}F₄ particles for varying Gd³⁺ doping concentrations;

FIG. 14 depicts the experimental setup used to demonstrate photocatalytic activity of IR-activated photocatalytic systems;

FIG. 15 (a) depicts gas evolution (e.g. CO₂) during photocatalysis, and FIG. 15 (b) depicts the change in pH of methyl red solution;

FIGS. 16(a) and (b) depict absorption spectra of aqueous solutions of methyl red collected at different times during photocatalytic reactions;

FIGS. 17 (a) and (b) depict absorption spectra of aqueous solutions of methylene blue collected at different times during photocatalytic reactions; and

FIG. 18 depicts a corrugated macrostructure according to one embodiment of the present invention.

### DETAILED DESCRIPTION OF PREFERRED EMBODIMENTS

The present invention provides photoelectric systems that utilize solar energy more efficiently by exploiting photon wavelengths more available in the solar spectrum than UV radiation. The photoelectric systems of the present invention enhance the performance of photo-catalytic and photovoltaic technologies by reducing light transfer limitations (e.g. scattering and absorption losses). In place of UV radiation, lower energy radiation is used to activate the photo-electric system of the present invention. Besides low scattering losses and more efficient light transfer, another benefit of using lower energy radiation such as IR is its deeper penetration depth in various systems (e.g. water, organic solvents and biological tissue). Deeper penetration enables activation of semiconductors embedded deep within or supported by porous structures and matrices.

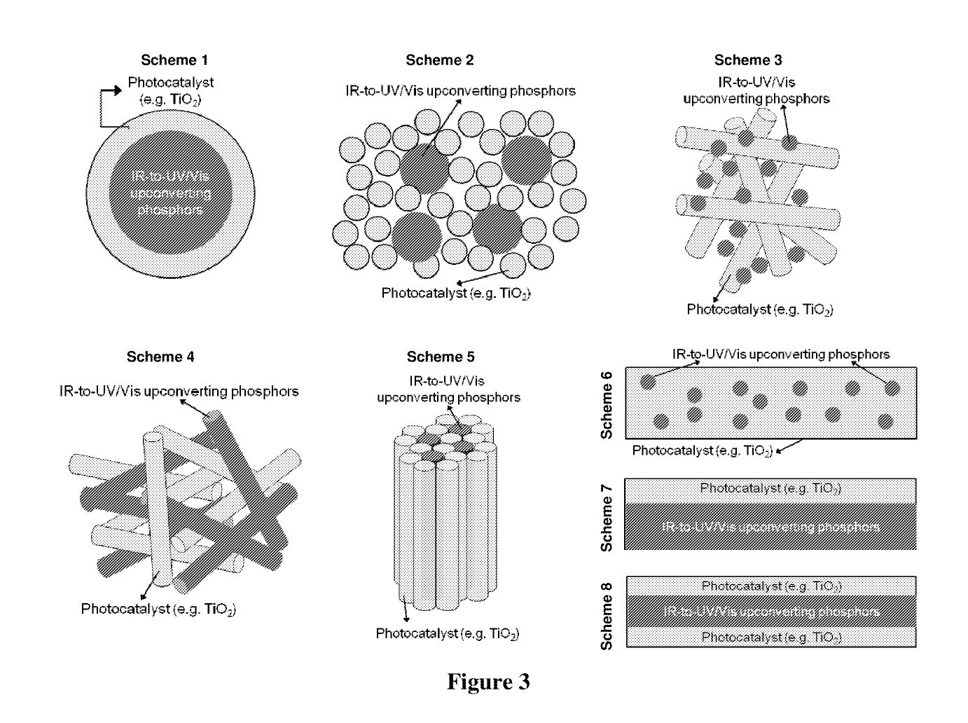

Lower energy activation using IR radiation is accomplished through the integration of upconverting rare earth doped phosphors with a semiconductor photocatalyst (e.g. TiO₂) as shown in FIG. 3. Upconverting rare earth doped phosphors convert low photon energy IR radiation into effective high photon energy UV emissions. The UV light is localized to the vicinity of the semiconductor to enhance photocatalytic performance. Energy transfer and UV emission is limited to the fine length scales of the microstructure of upconverting rare earth doped phosphors and TiO₂ and not the macroscopic length scales where UV emission could pose a safety hazard.

The absorption and emission properties of rare earth doped phosphors can be tailored by controlling the local environment, such as site symmetry, crystal field strength and electron-phonon interaction strength of rare-earth dopants. Suitable host compounds, rare earth dopants and methods of making phosphor compounds are disclosed by U.S. Pat. Nos. 6,699,406 and 7,094,361, the disclosures of which are incorporated herein by reference. Halide hosts (e.g. NaYF₄, YF₃, LaF₃) are favored for its low phonon energies which minimize non-radiative losses to enable intense upconverting emissions.

Assuming only absorption losses the maximum separation distance between upconverting rare earth doped phosphors and semiconductor photocatalysts can be determined by the reciprocal of the matrix's absorption coefficient, αₘₐₜᵣᵢₓ in the ultraviolet region, wherein the maximum separation distance=1/αₘₐₜᵣᵢₓ(λ). Table 2 shows an example of the maximum separation distance when the IR-activated photoelectric systems are dispersed in the different matrices. The experimentally determined maximum separation distance may differ from the values as listed in Table 2 as a result of other factors (e.g., scattering losses, variations in chemical composition of medium) that can lead to further optical losses. Regardless, the maximum separation distance can be determined by one of ordinary skill in the art without undue experimentation guided by the present specification. For systems where scattering losses dominate (e.g. large particles, large refractive index mismatch), the maximum separation distance will be shorter than that shown in Table 2. Using Rayleigh's scattering theory as an approximation, maximum separation distance where scattering losses dominate will be

$${d}_{UCP \text{-} SC} ∼ - {\frac{{λ}^{4}}{32 {π}^{4} {φ}_{p} {xr}^{3} {n}_{m}^{4}} [ \frac{{( {n}_{p} / {n}_{m} )}^{2} + 2}{{( {n}_{p} / {n}_{m} )}^{2} - 1} ]}^{2} ,$$

where λ is wavelength of light, φₚ is volume fraction of particles, r is particle size, nₚ is refractive index of inorganic particle and nₘ is refractive index of matrix. In this case, comparing the maximum separation distance at 350 and 900 nm with respect to that at 300 nm, differences in penetration depths are

$$\frac{{d}_{UCP \text{-} SC , 350}}{{d}_{UCP \text{-} SC , 300}} = {( \frac{350}{300} )}^{4} = 1.8 , \\ and$$

$$\frac{{d}_{UCP \text{-} SC , 900}}{{d}_{UCP \text{-} SC , 300}} = {( \frac{900}{300} )}^{4} = 81 ,$$

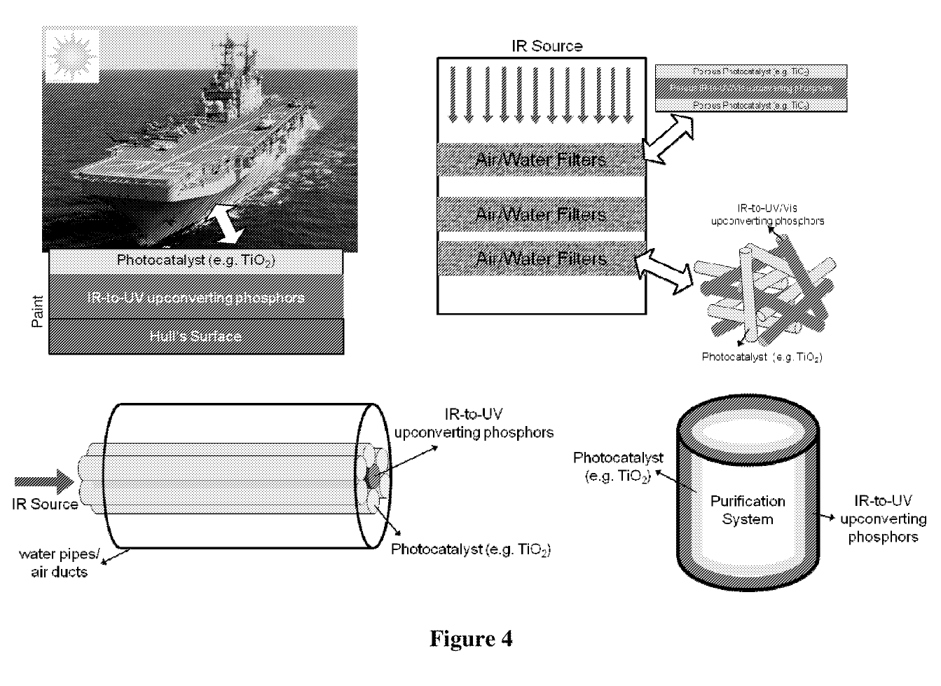

respectively. Examples that illustrate how the various schemes can be incorporated with different potential engineering applications are shown in FIG. 4. Because the maximum separation distance depends on the optical properties of the medium in which the system will be immersed, the microstructure and hierarchy of the photoelectric system is engineered accordingly, as shown in FIG. 3.

That is, upconverting rare earth doped phosphors and semiconductor photocatalysts (e.g. TiO₂) at different length scales (nano-, micro- and macroscales) and forms (e.g. non-porous or porous powders, films, or monoliths) can be integrated together according to the various arrangements in FIG. 3. Each of the schemes shown in FIG. 3 can be dispersed or deposited in various matrices (e.g. air or water) and supports (e.g. stainless steel, glass, polymers) or function alone without a matrix or other support.

### Scheme 1—Core-Shell Particles

The upconverting rare earth doped phosphor and semiconductor photocatalyst are inte-grated in the form of a core-shell microstructure 1. The semiconductor photocatalyst forms a continuous shell layer 3 covering the upconverting rare earth doped phosphor in the core 5. The core-shell microstructure is not limited to only particles with a spherical morphology, and can be further extended to apply to essentially any morphology, including, but not limited to, platelets, prisms, rods, fibers and cubes. The semiconductor photocatalyst shell layer may be porous or non-porous. Having a UV absorbing semiconductor layers (e.g. TiO₂) surrounding upconverting rare earth doped phosphor cores also prevents the escape of undesirable and hazardous UV emissions from the photoelectric system.

Several methods can be used to coat phosphors of varying morphology with photo-catalysts, using chemistry that can vary based on (a) solvent type, (b) precursor concentration and type, (c) surface capping agent (with or without), (d) reaction temperature and (e) other additional processing steps (e.g. calcination or sintering). Various types of chemistries can be utilized as well, such as: sol-gel, heterogeneous precipitation, particle impregnation, seeding methods etc. One of ordinary skill in the art will understand how to coat phosphors of varying morphology with semiconductors without undue experimentation.

The reaction activity of the photoelectric systems described in this scheme is controlled by a number of microstructure variables, including particle size, aspect ratio, shell thickness, volume ratio of phosphor to semiconductor and interfacial area between core and shell. While small particle sizes favor photoelectric activity because of an increase in specific surface areas, the trade-off is lower emission intensities from the upconverting rare-earth doped phosphors. Thus microstructure variables are preferably controlled to optimize reaction activity of the photo-electric system that adopts this scheme.

### Scheme 2—Mixture of Particles Having Similar Aspect Ratios

A mixture of both rare earth doped phosphors and semiconductor photocatalysts having either ordered or disordered arrangements can be employed as a photoelectric system according to the present invention. The rare earth doped phosphor and semiconductor photocatalyst particles have one of the following morphologies: spheres, rods, tubes, prisms, platelets, fibers, cubes, and the like. The mixture can be further compacted to form a solid pellet or tablet using conventional ceramic pressing technologies (e.g. hydraulic press and hot press). Alternatively, the mixtures can be dispersed in another external matrix (liquid or gas) or supported on a porous solid matrix (e.g. zeolites or fiber networks). For this scheme the distance between the upconverting rare earth doped phosphors and semiconductors should be maintained below the maximum separation distance as shown in Table 2. Considering the separation distances shown in Table 2, particle sizes can cover the nano-, micro- and macro-regimes. Further control of the photo-electric systems described in this scheme can be achieved by manipulating the volume ratio and particle sizes of upconverting rare earth doped phosphors and semiconductors.

### Scheme 3—Mixture of Particle and Rods, Fibers or Hollow Tubes of Semiconductors

Scheme 3 is an extension of Scheme 2 to include mixtures of upconverting rare earth doped phosphors and semiconductor photocatalysts each comprising of different particle morphologies. An example is shown in FIG. 3 where spherical upconverting rare-earth doped particles were mixed with semiconductors in the form of rods, fibers, tubes, and the like. As with Scheme 2, the mixture can be either compacted to form a solid pellet or tablet, or dispersed in another external matrix (liquid or gas) or be supported on a porous or non-porous solid matrix (e.g. zeolites, stainless steel). A critical distance should be maintained between the upconverting rare earth doped phosphors and semiconductors below the maximum separation distance as shown in Table 2. Considering the separation distances shown in Table 2, particle sizes can cover the nano-, micro- and macro-regimes. Further control of the photoelectric systems described in this scheme can be achieved by manipulating the volume ratio and particle sizes of upconverting rare earth doped phosphors and semiconductor.

TABLE 2
Maximum separation distance, d_{ucp-sc} (λ), between upconverting rare earth doped
phosphors and semiconductor photocatalyst

| | λ | | α | d_{ucp-sc} | λ | | α | d_{ucp-sc} | λ | | α | d_{ucp-sc} |
|---|---|---|---|---|---|---|---|---|---|---|---|---|
| Materials | (nm) | Abs. | (cm⁻¹) | (cm) | (nm) | Abs. | (cm⁻¹) | (cm) | (nm) | Abs. | (cm⁻¹) | (cm) |
| Water | 200 | 0.010 | 0.023 | 43.4 | 300 | 0.005 | 0.012 | 86.8 | 400 | 0.005 | 0.012 | 86.8 |
| Acetone | 330 | 1.000 | 2.303 | 0.434 | 350 | 0.010 | 0.023 | 43.4 | 400 | 0.005 | 0.012 | 86.8 |
| 2-Propanol | 205 | 1.000 | 2.303 | 0.434 | 300 | 0.005 | 0.012 | 86.8 | 400 | 0.010 | 0.023 | 43.4 |
| Benzene | 278 | 1.000 | 2.303 | 0.434 | 300 | 0.020 | 0.046 | 21.7 | 400 | 0.005 | 0.012 | 86.8 |
| Chloroform | 245 | 1.000 | 2.303 | 0.434 | 300 | 0.005 | 0.012 | 86.8 | 400 | 0.005 | 0.012 | 86.8 |

### Scheme 4—Interpenetrating Network of Both Fibers

The photoelectric system of the present invention can also be composed of an interpenetrating network mesh of upconverting rare earth doped phosphors and semiconductor fibers. The fibers can be porous or non-porous. Methods by which the rare earth doped phosphors and semi-conductors may be formed into an interpenetrating network of fibers are disclosed, for example, by Neukam, et al., Mater. Sci. Forum, 631-632, 471-476 (2010) and Mattern, A. et al., J. Eur. Ceram. Soc., 24(12), 3399-3408 (2004), the disclosures of which are incorporated by reference.

The interpenetrating network of fibers may be applied to current engineering applications without further modification or support (e.g. 2-dimensional planar sheet of interpenetrating fibers), immersed in a liquid (e.g. water) or gas (e.g. air), or be supported on solids (e.g. spun together with cotton or nylon fibers). The activity of such photoelectric systems is governed by the volume ratios, sizes and aspect ratios of the fibers of the up-converting rare earth doped phosphors and semiconductors. Critical distances should be maintained between the upconverting rare earth doped phosphors and semiconductors to be below the maximum separation distance as shown in Table 2. Considering the separation distances shown in Table 2, particle sizes can cover the nano-, micro- and macro-regimes.

### Scheme 5—Ordered Fibers or Hollow Tubes Structures

As a modification to Scheme 4, fibers or tubes of upconverting rare earth doped phosphors and semiconductors can be arranged to have an ordered configuration. An example of such an ordered arrangement of upconverting rare earth doped phosphor and semiconductor fibers is shown in FIG. 3, where the fibers are aligned and arranged in a close-packed manner. Methods by which fibers or tubes of rare earth doped phosphors and semiconductors can be arranged in an ordered configuration are disclosed, for example, by Padture, et al., Ceramics and Single-Walled Carbon Nanotubes. Advanced Materials (Weinheim, Germany), 21(17), 1767-1770 (2009) and Wang, Treatise on materials science and technology: Ceramic Fabrication Processes, V9, Acad. Press: Orlando, Fla. 1976, the disclosures of which are incorporated by reference. These fibers can be porous or non-porous and of a sub-micron length scale. The fibers may be applied without further modification or support as a bundle immersed in a liquid (e.g. water) or gas (e.g. air), or be supported on solids. Volume ratios, sizes and aspect ratios of these fibers will control the semiconductor activity of the photoelectric systems.

### Scheme 6—Particles Dispersed in Matrix of Photocatalyst

In another configuration according to the present invention, upconverting rare-earth doped phosphors can be embedded within a continuous matrix of the semiconductor. The semiconductor matrix can be porous or non-porous with various geometries and forms such as spheres, cubes, rods, tubes, prisms, films, sheets and the like. Methods by which rare earth doped phosphors may be embedded within a continuous matrix of a semiconductor are disclosed, for example, by Xiang, et al., Nuclear Instruments & Methods in Physics Research, Section B: Beam Interactions with Materials and Atoms, 268(9), 1440-1445 (2010). Kim et al. Applied Catalysis, B: Environmental, 84(1-2), 16-20 (2008). Zhang, et al. J. Mater. Process. Tech, 197(1-3), 31-35 (2008) and Wang, Treatise on materials science and technology: Ceramic Fabrication Processes, V9, Academic Press: Orlando, Fla. (1976), the disclosures of which are incorporated by reference. The solids loading and sizes of upconverting rare-earth doped phosphors are contributing factors that will influence the semiconductor activity of this IR-activated photoelectric system.

### Scheme 7—Continuous Bi-Layer Structure and Coatings

A continuous layer of the semiconductor can be coated onto a film or sheet of upconverting rare earth doped phosphors forming a continuous bi-layer structure as shown in FIG. 3. Methods by which a continuous layer of a semiconductor can be coated onto a film or sheet of rare earth doped phosphors to form a continuous bi-layer structure are disclosed, for example, by Chen et al., Applied Catalysis B: Environmental, 62, 255-264 (2006) and Wang, Treatise on materials science and technology: Ceramic Fabrication Processes, V9, Acad. Press: Orlando, Fla. (1976), the disclosures of which are incorporated by reference. Each layer of material can be porous or non-porous. In this case, the semiconductor activity is governed by thickness and relative thickness of each layer.

### Scheme 8—Continuous Multilayer Structures and Coatings

A continuous multilayer structure and coating of upconverting rare earth doped phosphors and semiconductors are a further extension of scheme 7, obtained by laminating the bi-layer structures of Scheme 7 by conventional techniques. See, for example, Steele, et al., Current Opinion in Solid State & Materials Science, 2, 563-565 (1997) and Wang, Treatise on materials science and technology: Ceramic Fabrication Processes, V9, Acad. Press: Orlando, Fla. (1976). Having UV absorbing semi-conductor layers (e.g. TiO₂) surrounding the upconverting rare earth doped phosphors also prevents the escape of undesirable and hazardous UV emissions from the photoelectric system of the present invention. Similar to scheme 7, the semiconductor activity is governed by layer thickness and the relative thickness of each layer. An additional variable that can be used to tailor the catalytic activity is the number of layers.

In summary, the above-mentioned schemes describe various microstructures applicable to the IR-activated photoelectric systems of the present invention. The phosphor and semiconductor phases can be bonded by either primary (e.g. covalent, ionic, etc.) or secondary (e.g., van der Waals, hydrogen bonds, electrostatic, etc.) bonds. Various processing methods (e.g., sintering, sol-gel, etc.) can be employed to bond the phosphor and semiconductor phases. In addition, binders (e.g. polymers, cements, etc.) can be added to the mixture to facilitate the bonding between the phosphor and semiconductor phases. Careful selection of the type and concentration binder is required to ensure that the binder phase does not adversely affect the performance of the photoelectric material (e.g. optical losses from absorption and scattering caused by the binder, reduced reactive surface area for the semiconductor, etc.)

### Potential Applications of IR-Activated Photocatalyst Systems

Technological applications are provided that are based on the photoelectric systems of the present invention (see FIG. 4). Solar-powered IR-activated photovoltaic systems will enable the reduction in reliance of various technologies on external power sources (e.g. batteries). For example, by more efficiently harnessing solar power for water purification systems, less process-ing steps and subsequently less generated energy will be needed for water treatment. Besides being powered by solar energy, IR-activated photoelectric systems can also be activated using eye-safe, low cost, Hg-free and portable IR illuminators, lamps or photodiodes when ambient light is not available. Consequently, this can lead to the elimination of Hg-containing UV light sources. Moreover, chronic exposure of eyes to UV light has been known to cause cataracts. Because harmful UV rays are not used, IR-activated photoelectric systems can deployed and cover larger areas without causing inconvenience and interruption to work or daily activities. Furthermore, the photoelectric systems can be easily regenerated and reused. Key benefits and technological advancements are presented and described further below.

### Solar Power Generation

Current photovoltaic systems can be broadly categorized as semiconductor (inorganic) and organic photovoltaic cell systems. In semiconductor photovoltaic cells, photogenerated electrons and holes are collected on separate electrodes (e.g. p-type Si and n-type Si). Charge separation in a semiconductor occurs at p-n junctions or heterojunctions. Organic photovoltaic cells offer a low cost alternative to semiconductor photovoltaic cells. However, organic photo-voltaic cells operate at lower efficiencies due to the additional energy that is required for the dissociation of excitons (i.e. bound states of electron-hole pair) into free electrons and holes. The different organic photovoltaic cells that are currently being developed include: (1) polymer-fullerene-, (2) polymer-, (3) low-molecular organic-, (4) tandem (i.e. >1 heterojunctions)-, (5) hybrid (i.e. organic-inorganic composites)-, and (6) dye-sensitized solar cells.

The upconverting phosphor component can be integrated into either semiconductor or organic photovoltaic cells. In semiconductor photovoltaic cells, the upconverting phosphors can be incorporated in the form of a layered structure on the electrodes. In organic photovoltaic cells, the upconverting phosphors can be incorporated either within the polymer forming a hybrid composite or in the form of a coating on the electrodes.

### Chemical Waste Remediation

A wide variety of volatile organic chemical species (e.g. toluene, phenol, chloroform, benzene, etc.) in either liquid or gas phases can be removed using the photocatalytic systems of the present invention. Non-volatile organic species can also be remediated. The photoelectric systems of the present invention can be easily integrated and adapted into existing chemical waste treatment plants. For instance, fiber bundles of the IR-activated photoelectric system of the present invention can be included within pipelines delivering effluent waste streams. By utilizing efficient solar power to excite IR-activated systems, the present invention allows the reduction in reliance on external electrical power sources. Furthermore, non-toxic by-products such as CO₂ and N₂ are obtained during the photocatalytic degradation of organic chemical species.

### Self-Cleaning Applications

Non-volatile naturally occurring fatty acids (e.g., octadecanoic (stearic) acid, hexadecanoic (palmitic) acid, etc.) can be photocatalytically degraded on coatings of the photoelectric system of the present invention, thus enabling removal of oily finger-prints and organic residues to make surfaces easier to clean. Besides removing organic residues, surface properties (e.g. hydrophilicity and hydrophobicity) can be controlled using the photoelectric systems of the invention. The IR-activated photoelectric systems can be applied as coatings on surfaces for architecture (e.g. windows, building facades), automotive (e.g. rear view mirrors), office (e.g. computer screens) and appliances (e.g. stove tops, refrigerators, television), thus imparting self-cleaning properties to these objects. The photoelectric systems of the present invention can also be coated on the surfaces of the hulls of ships and heat exchangers to prevent or reduce fouling (e.g. barnacles, algae, protein precipitates).

The photoelectric self-cleaning coating systems of the invention can be provided with sensor-activated cleaning systems when ambient light is not available. For example, upon measuring a certain reduction in transmittance caused by dust accumulation, a sensor can activate an IR illuminator to activate the semiconductor to subsequently enable the cleaning of the surfaces is systems of the present invention in which the phosphorescent compound is a rare earth doped upconverting phosphor that upon excitation with IR wavelength photons emits UV wavelength photons capable of activating the titanium dioxide semiconductor.

### Sterile Coatings and Processing

Sterilization and removal of biological microbes can be achieved using photocatalysis. The photoelectric systems of the present invention can be coated onto various surfaces such as cooking utensils, surgical tools, medical devices, biomedical implants, food packages, door knobs and public and private rest room surfaces to allow easy and effective sterilization. Having door knobs and other frequently touched surfaces and objects (e.g. money, escalator handrails, elevator buttons) coated with IR-activated photoelectric systems will allow these surfaces and objects to remain sterile and subsequently prevent transmission of contagious diseases.

The photoelectric systems disclosed herein can also be integrated into aseptic processing lines required in food processing and pharmaceutical plants to enable sterile processing and packaging. Using this innovation, deeper sterilization can be achieved.

Furthermore removal of fouling agents from implanted medical devices can be completed without the need of undergoing surgical procedures when the phosphorescent compound is a rare earth doped upconverting phosphor that upon excitation with IR wavelength photons emits UV wavelength photons capable of activating the titanium dioxide semiconductor. Non-invasive, deep tissue penetrating IR light can be used to trigger IR-activated photoelectric systems coated on medical devices and implants that will subsequently enable removal of any undesirable foul-ing protein deposits that would otherwise impede performance of the implanted medical devices.

### Ozone Generation

The high electrical demands of current ozone generators (e.g. VUV lamps, corona discharge tubes) have limited the use of ozone for purification and sterilization. As an extension and modification of the sterile coating and processing embodiments disclosed herein, the photo-electric system of the present invention can be developed into a low cost ozone generating system. The high oxidation potential of ozone can be used to remove pesticide residues by severing carbon-carbon bonds, and kill microorganisms in air and water.

### Water Purification and Treatment

The photoelectric systems of the present invention can be easily integrated and adapted into existing water treatment plants and portable water purification systems. Systems in which the phosphorescent compound is a rare earth doped upconverting phosphor that upon excitation with IR wavelength photons emits UV wavelength photons capable of activating the titanium dioxide semiconductor employ solar power for energy efficiency, allowing either the removal or reduction in the reliance and dependence on external electrical power sources (e.g. batteries). For instance the IR-activated photoelectric systems of the invention can be included within water pipelines or within large water tanks and portable water carriers to decompose low concentra-tions of organic impurities in the form of organic compounds, organic matter, and the like.

### Air Purification and Treatment

Another application for photoelectric systems of the present invention is the purification of both indoor and outdoor air, including the removal of impurities in the form of organic com-pounds, organic matter, and the like. The photoelectric systems disclosed herein can be incorporated into current air filtration (e.g. on the filters) and circulation (e.g. on fans or surfaces of air vents) units found in office buildings, hospitals, vehicles (e.g. cars, army tanks, airplanes), toilets and confined places to enable indoor air purification. Indoor air purification can be achieved by employing a sensor-activated device that turns on an illuminator, lamp, photodiode, and the like, adapted to excite the phosphorescent compounds to activate the semiconductors in the filters to allow air purification.

When the phosphorescent compound is a rare earth doped upconverting phosphor excited by IR wavelength photons, IR radiation can be used instead of UV radiation to activate the photoelectric system. Because harmful UV rays are not used, the IR-activated photoelectric systems can be deployed and cover much larger areas without causing inconvenience and interruption to work or daily activities.

For outdoor air purification, the photoelectric systems of the present invention can be incorporated into existing architectures (e.g. roof tiles), air systems in automobiles or vehicle exhaust systems. When the phosphorescent compound is a rare earth doped upconverting phosphor excited by IR radiation, photoelectric systems incorporated on building facades and roofs instead of catalytic converters in automobiles will enable solar powered air purification.

### Fuel and Energy Generation

The photoelectric system of the present invention can be exploited for the efficient generation of electrical power (e.g. dye-sensitized solar cells) and hydrogen production. Enabling efficient hydrogen production will lead to significant advancements in the generation of re-usable energy. Up to now, hydrogen generation has been inefficient primarily due to its high energy demands (e.g. electrical). When the phosphorescent compound is a rare earth doped upconverting phosphor excited by IR wavelength photons, IR-activated photoelectric systems that harness solar energy for hydrogen production are provided that reduce the demand for energy from non-renewable resources to generate hydrogen. Using the IR-activated photoelectric systems disclosed herein will improve the efficiency of solar powered energy generation and hydrogen production by reducing light transfer limitations and enhancing electron injection rates by converting unused low energy photons to useful high energy photons.

Besides generating hydrogen, other fuel sources such as methane, methanol and formaldehyde can be generated from sources like biomass (e.g. lignocelluloses) and carbon dioxide. Carbon dioxide emissions from industrial and combustion processes are the largest contributor among greenhouse gases. Besides converting undesired carbon dioxide into more useful compounds (e.g. methanol, methane), this technology will enable a method of reducing carbon dioxide that is less energy-consuming compared to other conventional fuel generation methods. Some of the factors in fuel generation that can affect photocatalytic performance are wavelength of ultraviolet light, pressure, temperature, solvents (water, acetonitrile, isopropanol) and moisture content (i.e. carbon dioxide to water ratio). Using the IR-activated photocatalyst system of the present invention will improve the efficiency of solar powered fuel generation by reducing light transfer limitations.

### Biomedical Applications

When the phosphorescent compound is a rare earth doped upconverting phosphor excited by IR wavelength photons, IR-activated photoelectric systems are provided by the present inven-tion for photodynamic therapy (PDT) cancer treatment by exploiting the strong oxidizing power of activated semiconductors to kill tumor cells. The semiconductors replace conventionally used PDT photosensitizers, such as porphyrins. PDT is a minimally invasive treatment that destroys target cells in the presence of oxygen when visible light irradiates a photosensitizer, generating highly reactive singlet oxygen that then attacks the cellular target. The use of photosensitizers excited by visible light has thus far limited the use of PDT to tissues accessible with a light source. Current clinical applications include the treatment of solid tumors of the skin, lungs, esophagus, bladder, head, neck, and the like.

Photoelectric systems according to the present invention generate tumor-destroying free radicals without the need for visible light. IR-light, which deeply penetrates tissues, can be used to excite the phosphor and activate the semiconductor to generate the peroxo complexes. The photoelectric systems can be injected at the site of the disease or couples to ligands that target the position of the cancer cells for systemic delivery. For a review of current clinical applications for PDT to which the photoelectric systems of the present invention can be readily adapted see, Celli et al, Chem. Rev., 110, 2796-2838 (2010), the disclosure of which is incorporated by reference.

In addition to PDT, the free radicals generated by the photoelectric systems of the present invention can be employed in other therapeutic applications. For example, the free radicals gen-erated by photoelectric semiconductors can be used to kill or slow the growth of tumor cells, hematological malignancies, and other undesirable cell growths, malignant or benign. Pathogenic viruses, bacteria, fungi, parasites and prions can also be killed, or their growth slowed. The proliferation of hyper-active immune system cells in patients suffering from an auto-immune or inflammatory disease can also be effectively suppressed. The free radicals generated can also be used to break down toxins and allergens responsible for inflammation, kidney disease, liver disease, and the like.

The oxidizing power of the activated semiconductors can also be paired with the redox properties of CeO₂ to enable cell or nerve regeneration. It has been observed that depending on the redox properties of the activated semiconductor, the free radicals generated either kill undesirable cells or regenerate desirable cells and tissues. See, for example, Dasa et al., Biomater., 28, 1918 (2006).

The IR-activated photoelectric systems of the invention thus offer a non-invasive surgical approach to disease treatment.

### IR-Activated Photoelectric Systems

### UV-Emitting Phosphors Excited by IR Radiation

The nanoparticles can be prepared from essentially any optically transparent inorganic material capable of being doped with one or more active ions. Suitable inorganic materials include ceramic materials such as oxides, halides, oxyhalides and chalcogenides of metals such as lanthanum (La), lead (Pb), zinc (Zn), cadmium (cd), and the Group II metals of the Periodic Chart, e.g., beryllium (Be), magnesium (Mg), calcium (Ca), strontium (Sr) and barium (Ba). Group III metal ceramics can also be used, such as aluminosilicates.

The active ions are typically rare earth elements. However, essentially any ion that will absorb IR wavelengths and emit in either the UV or visible spectra can be used. In the present invention, the active ions entirely reside in individual low-phonon energy materials. Energy level analyses for various rare earth dopants are shown in FIG. 5. While all rare earth doped host materials emit UV photons upon excitement with IR radiation, which thus may be used alone or in combination in the present invention, upconversion is particularly strong in host systems doped with Yb, Tm, Er and Gd, examples of which include NaYF₄:Yb—Tm, NaGdF₄:Yb—Tm LaF₃:Yb—Tm, YF₃:Yb—Tm, GdF₃:Yb—Tm, YF₃:Yb—Gd—Tm and NaYF₄:Yb—Er phosphors.

### Semiconductors Activated by UV or Visible-Radiation

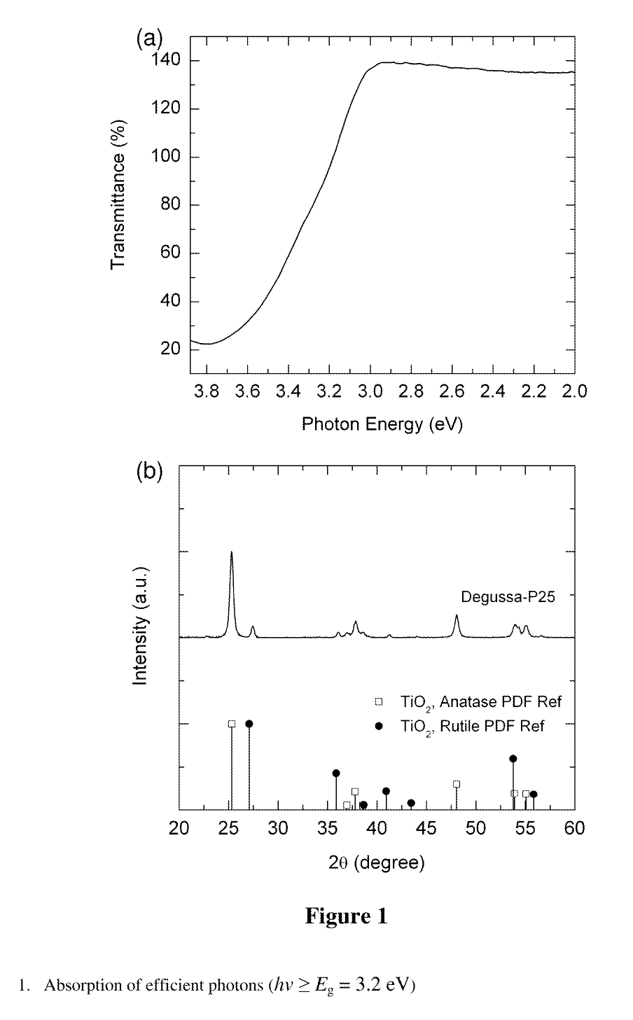

Semiconductors suitable for use with the present invention are activated by UV or visible radiation and include binary oxides (e.g., anatase TiO₂, rutile TiO₂, CeO₂, ZnO, Fe₂O₃, WO₃, Ta₂O₅, VO₂, etc.), ternary and quaternary metal oxides (e.g., K₄Nb₆O₁₇, HCa₂Nb₃O₁₀ or KCa₂Nb₃O₁₀, K₄Ce₂Ta₁₀O₃₀, K₄Ce₂Nb₁₀O₃₀, LiTaO₃, NaTaO₃, KTaO₃, SrTiO₃, Sr₃Ti₂O₇, La₂Ti₂O₇, NaTi₂O₄(OH)₂, K₂La₂Ti₃O₁₀, K₄Ce₂Ta₁₀O₃₀, K₄Ce₂Nb₁₀O₃₀, etc.), metal sulfides (e.g., ZnS, CdS, CdₓZn₁₋ₓS, etc.), nitrides, oxynitrides and oxysulfides (e.g., TaON, Sm₂Ti₂S₂O₅, (Ga₁₋ₓZnₓ)(N₁₋ₓOₓ), etc.), and the like. Anatase TiO₂ is particularly favored in most photochemical reactions. (See FIGS. 1(a) and 1(b).) The crystal structure and order of the semiconductor is critical to its photoelectric performance and the rate constant of anatase is ˜25 times that of amorphous TiO₂. See, Zhao et al., J. Mater. Chem. 20(37), 7990-7997 (2010).

Photoelectric Systems with More than One Type of Phosphorescent Materials

The IR-activated composite can include more than one type of phosphorescent material having varying rare earth dopants and hosts, wherein each type of phosphor can be excited with a different wavelength of infrared light to generate the same or different wavelength(s) of UV or visible emissions. These phosphors having various possible sizes and morphologies can be mixed at equal amounts or varying ratios.

### Particle Morphologies

The rare earth doped phosphors and semiconductors can consist of essentially any particle morphology not subject to any limitations disclosed herein for Schemes 1-8. Examples of suitable particle morphologies include, but are not limited to, regular geometries such as cubes, rectangular solids, cuboids, prisms, discs, pyramids, complex polyhedrons, multi-faceted particles, cylinders, spheres, cones, and the like; hollow structures such as rings, tubes, and the like; and irregular particle shapes, such as acicular, angular, bent, channeled, concave, crescent, columnar, dendritic, equant, euhedral, fibrous, flaked, flattened, fractal, glass-like, grape-like, granular, irregular, layered, long-thin, lumpy, lath, modular, needle, oblong, plate, platelet, potato, ribbon, rippled, rod, rounded, shard, sheet, smoothed, eraser, burrito, Africa, jelly fish, worm, subhedral, striated, subangular, subsphere, twisted, and the like. For examples of these and other particle morphologies and methods by which they can be made, see Allen, Particle Size Measurement (Third Ed. Chapmen and Hall, New York 1981); Merkus, Particle Size Measurements: Fundamentals, Practice, Quality (First ed. Springer 2009), http://www.nist.gov/lis-pix/doc/particle-form/part-morph-gloss.htm#slide67, http://mathworld.wolfram.com/Poly-hedron.html and http://www.onlinemathlearning.com/solid-geometry.html), the disclosures of which are incorporated by reference.

Photoelectric Systems with More than One Type of Semiconductor

The IR-activated photoelectric system can comprise more than one type of semiconductor (i.e. amorphous or crystalline, crystal phase, chemical composition), wherein each type of semi-conductor can have either different chemical reactivities (e.g. reaction rates, selectivity) or have different absorption wavelengths (UV or visible). For example, the semiconductor choice can include both a fast-reacting and a slow-reacting semiconductor material to enable a constant rate of reaction as well as the continued use of the composite system over longer periods of time. While the fast-reacting semiconductor material will enable a fast response, it will be reach its maximum reaction capacity much more quickly. On the other hand, the slower reacting semi-conductor material will reach its maximum reaction capacity more slowly to enable the continued use of the system for a longer duration.

### Macrostructure Fabrication

The macrostructure of the photoelectric devices of the present invention is just as critical as the microstructures of the semiconductor and phosphor particles they contain. Yet, essentially any macrostructure is feasible, provided that it allows the phosphor particles to be mixed with the semiconductor particles on a scale of homogeneity so that adequate separation distance between the phosphor and semiconductor photoelectric material is maintained. The photoelectric systems can be processed to prepare porous (e.g. cellular-) or partially or fully dense monolithic-ceramic macrostructures. Monolithic macrostructures can be used as formed, subsequently sintered or processed in a variety of ways to bond the semiconductor and phosphor phases using chemical vapor deposition, sol-gel, hot isotatic pressing, hot pressing, spark plasma sintering, as well as conventional sintering of powders. One of ordinary skill in the art guided by the present specification will be able to apply their knowledge on processing to form dense structures without undue experimentation.

The following sections describe examples of methods to form porous cellular structures. However, one of ordinary skill in the art will recognize that the present invention is not limited to only these methods. Cellular ceramics have a wide range of forms: foams, honeycombs, corrugated structures, interconnected rods, interconnected fibers, high density closed-cell structures, and the like. See for example, Colombo, Phil. Trans. R. Soc. A, 364, 109-124 (2006) and Wadley, Phil. Trans. R. Soc. A, 364, 31-68 (2006). Cellular ceramics are a class of materials containing a high level of porosity (>60 vol %) that are characterized by the presence of “cells” that are arranged three-dimensionally. The “cell” is an enclosed empty space possessing faces and solid edges, where the faces can either be fully solid or void, to give a closed- or open-cell material, respectively. The cells can be regular or exhibit random or graded variations in size, shape and distribution. Reticulated ceramics are open-cell materials consisting of interconnected voids surrounded by a web of ceramic ligaments. Processing routes strongly influence the macro- and micro-structure characteristics of cellular ceramics that in turn control performance and properties.

### Foam

Three different approaches can be followed to produce ceramic foams which comprise of cell walls that are randomly oriented in space: (1) replication of a sacrificial foam template, (2) direct foaming of a liquid slurry and (3) burn-out of pore formers.

### Honeycomb

Large dimension honeycombs comprising parallel prismatic cells are manufactured by paste extrusion of a variety of ceramic powders. After extrusion, further processing steps like drying, de-binding, and sintering may be needed. Honeycombs can be extruded with well defined, unidirectional channels with a triangular, square, circular, hexagonal-shaped cross-section, yielding a high permeability throughout the longitudinal direction of the component. Honey-comb structures can also be obtained by assembling lower dimensional parts, like hollow rods or sheets. Multicomponent systems can be formed using co-extrusion techniques to obtain either randomly mixed multicomponent extrudate or a ordered structure such as a core (phosphor) shell (semiconductor) extrudate.

### Corrugated Structure

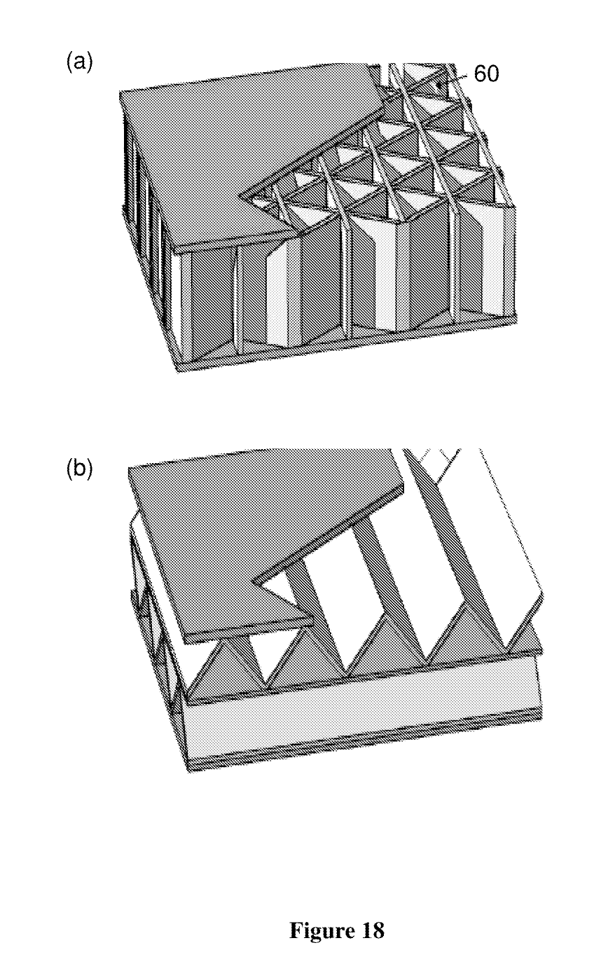

Corrugated structures are another class of cellular ceramics, similar to the honeycomb structures (see FIG. 18). The difference between the corrugated and honeycomb structures is in the three dimensional arrangement of the cell layer. In corrugated structures, each cell layer is aligned at a 90 degree angle with respect to each other.

### Interconnected Rods

Three-dimensional, periodic, cellular structures comprising of interconnected rods (cylindrical or with other shapes) can be produced by methods such as fused deposition, robocasting, 3-dimensional solid printing and many other robotic rapid prototyping techniques for making shaped ceramics. All these processing methods involve the patterning of extruded materials by using computer-aided design and build software programs to form complex architectures. Therefore, besides forming interconnected rod structures, other complex architectures (e.g. interconnected fibers, honeycomb etc.) can also be prepared using this method. The various methods use a wide range of feedstocks, the primary distinction between the fused and robotic deposition approaches is that fused deposition utilizes particle-filled, polymeric feedstocks, while robotic deposition utilizes concentrated colloidal gels as inks. The process control and materials forms obtained by either technique depend on rheological properties of the extrudate.

### Interconnected Fibers

Ceramic fiber mats can be formed using a wide range of methods. For example ceramic fiber mats are formed by collecting fibers that are randomly oriented in length and width in the direction of a moving conveying belt. The structure is built up in the thickness direction by adding layers of such deposits. These materials can also be termed as an “open-interconnected network.”

### Low Density Closed-Cell Structures

Another approach for producing cellular ceramics is the sintering of hollow spheres (or other shapes) to yield closed-cell structures. Hollow spheres are generally fabricated by nozzle, sacrificial core processes or sol gel techniques. After packing the spheres in a mold, they are joined together using a slurry coating, followed by sintering. The key benefit of these closed-cell structures is the ability to obtain highly buoyant lightweight structures due to its high porosity. This will enable the application of the IR-activated photoelectric systems in the form of materials that are buoyant on various liquids. This has utility for the removal of pollutants (e.g. oil spills, chemical spills, algae) that reside on surfaces of large bodies of water (e.g., lakes, oceans, seas, etc.) or other types of fluids.

### EXAMPLES

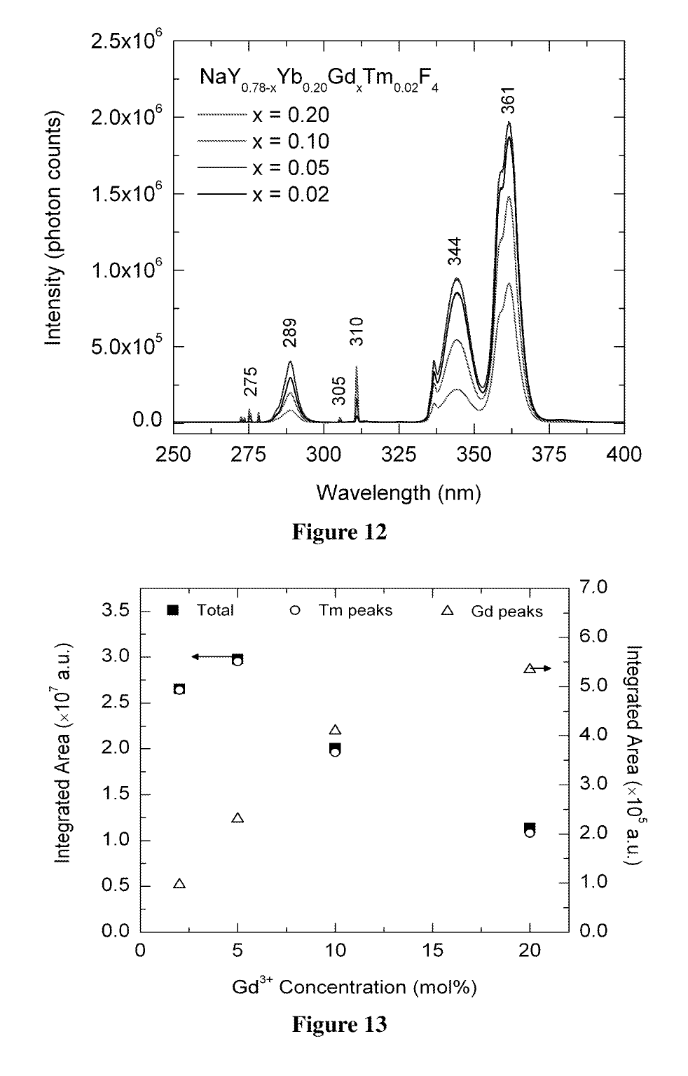

In the present application, IR-to-UV upconversion is demonstrated using as-synthesized NaYF₄:Yb—Er, NaYF₄:Yb—Tm and NaYF₄:Yb—Gd—Tm phosphors. (See FIGS. 12-13.) However, the demonstrated upconversion is produced to varying degrees by any rare earth doped system. Accordingly, the present invention extends to the use if essentially any rare earth doped host material in the photo-electric systems of the present invention. While thermal treatment of as-synthesized phosphors to obtain the IR-to-UV upconverting phosphors is reported to be necessary in the prior art, no thermal treatment was used for preparing the phosphors demonstrated here. Thermal treatment of the phosphors can facilitate and will be beneficial to further enhancements of photoelectric material performance.

### Characterization

Powder x-ray diffraction (XRD) patterns were obtained with a resolution of 0.04°/step and 2 sec/step with the Siemens D500 (Bruker AXS Inc., Madison, Wis.) powder diffractometer (40 kV, 30 mA), using Cu K_{α} radiation (λ=1.54 Å). Powder diffraction files (PDF) from International Centre for Diffraction Data (ICDD, Newtown Square, Pa.) PDF#97-017-2914, PDF#97-006-6650 and PDF#97-005-1917 for anatase TiO₂, rutile TiO₂ and hexagonal NaYF₄, respectively was used as reference.

The phosphor powder samples were packed in demountable Spectrosil® far UV quartz Type 20 cells (Sturm Cells, Inc, Atascadero, Calif.) with 0.5 mm path lengths for optical emission measurements. The emission spectra of nanoparticles excited at ˜976 nm with a 2.5 W laser (BW976, BW Tek, Newark, N.J.), was collected using the FSP920 Edinburgh Instruments spectrometer (Edinburgh Instruments, Livingston, United Kingdom) that was equipped with a Hamamatsu R928P photomultiplier tube detector.

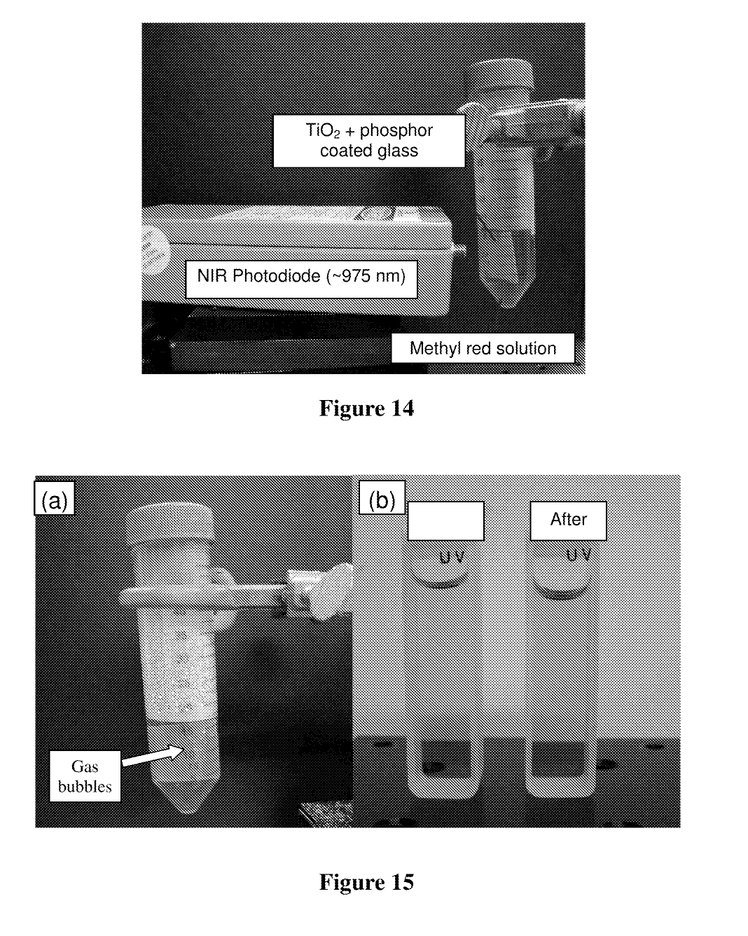

The absorption spectra of aqueous solutions of methyl red and methylene blue from 350 to 700 nm and 350 to 750 nm, respectively, were measured with a 4 nm slit and 1 nm step size, using a Perkin-Elmer Lambda 19 spectrophotometer (Perkin-Elmer, Waltham, Mass.) equipped with a 60 mm integrating sphere. The aqueous dye solutions were contained in a 3.5 mL quartz cuvette (Cole-Parmer, Vernon Hills, Ill.) which has a path length of 10 mm. (See FIG. 14.)

### Functional Testing of IR-Activated Photoelectric Systems

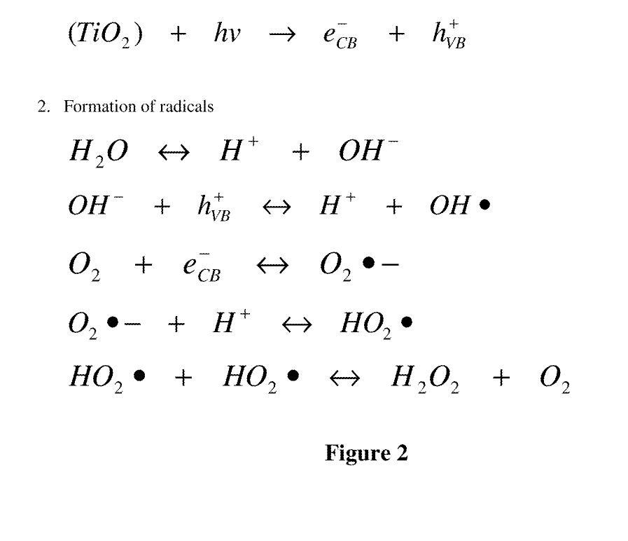

Waste waters generated by textile industries contain considerable amounts of non fixed dyes, especially azo-dyes (e.g. methyl red and methylene blue). Azo-dyes and their degradation products, such as aromatic amines, are highly carcinogenic. Photocatalytic dye degradation enables the conversion of these azo dyes into relatively safer chemicals like CO₂ and N₂. The chemical reactions for the degradation of various dyes are listed in Table 3. Typically, photo-catalytic degradation of azo dyes is monitored by measuring total organic carbon (TOC) content and chemical oxygen demand (COD). In addition to decrease in TOC and COD, the pH of the aqueous dye solution is expected to decrease during the photocatalytic process because of H⁺ evolution during dye degradation (see Table 3). Besides dye degradation, the pH of the aqueous solution is also governed by: water dissociation equilibrium (see FIG. 2), and the ionization state of organic dyes and their metabolites.

TABLE 3
Stoichiometric equation of dye total oxidation

| Dye | Chemical Equations |
|---|---|
| Methylene Blue | ${C}_{16} {H}_{18} {N}_{3} {S}^{+} + \frac{51}{2} {O}_{2} \to 16 {CO}_{2} + 3 {NO}_{3}^{-} + {SO}_{4}^{2 -} + 6 {H}^{+} + 6 {H}_{2} O$ |
| Orange G | C₁₆H₁₁N₂O₃S⁻ + 20O₂ → 12CO₂ + 2NO₃⁻ + SO₄²⁻ + 3H⁺ + H₂O |
| Alizarin S | C₁₄H₇O₇S⁻ + 14O₂ → 14CO₂ + SO₄²⁻ + H⁺ + H₂O |
| Methyl Red | ${C}_{15} {H}_{15} {N}_{3} {O}_{2} + \frac{43}{2} {O}_{2} \to 15 {CO}_{2} + 3 {NO}_{3}^{-} + 3 {H}^{+} + 6 {H}_{2} O$ |
| Congo Red | ${C}_{32} {H}_{22} {N}_{6} {O}_{6} {S}_{2}^{2 -} + \frac{91}{2} {O}_{2} \to 32 {CO}_{2} + 6 {NO}_{3}^{-} + 2 {SO}_{4}^{2 -} + 8 {H}^{+} + 7 {H}_{2} O$ |

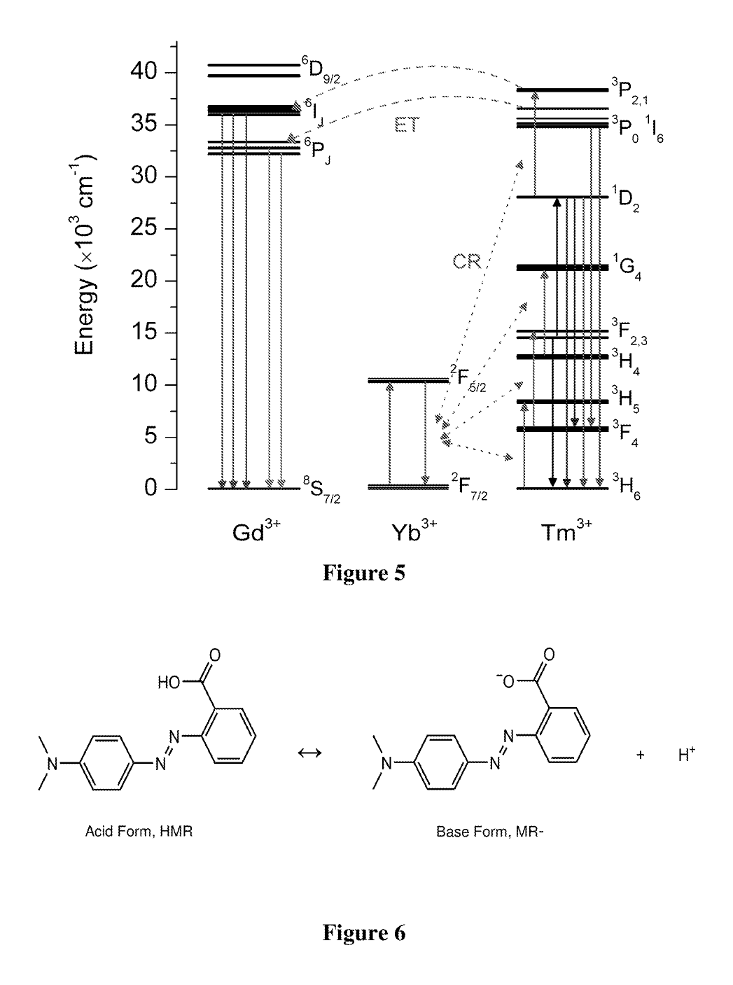

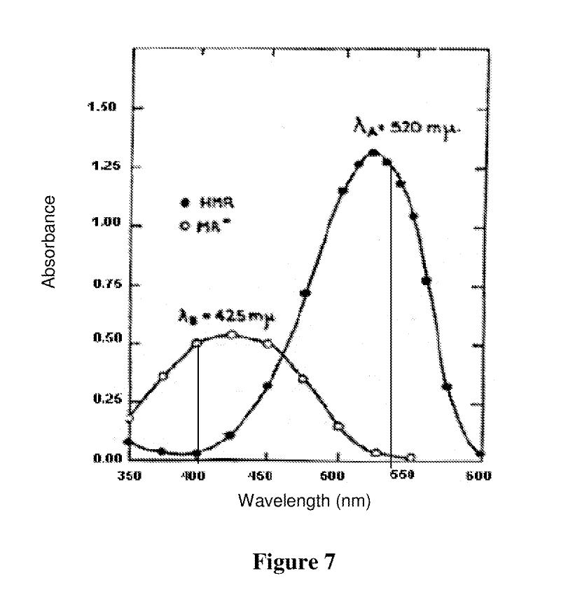

As one of the demonstration examples, the photocatalytic activity of the IR-activated photoelectric system was tested by monitoring the pH of saturated aqueous solutions of methyl red (MR). The equilibrium dissociation of aqueous solutions of protonated methyl red (HMR) is shown in FIG. 6. HMR (red) shows a maximum absorption at 520 nm, while the maximum absorption of MR anions (yellow) is at 425 nm (see FIG. 7). The relative intensities of each absorption peak will depend on equilibrium concentrations of HMR and MR anions in solution, which is determined by the solution pH. In this demonstration, the pH of the aqueous solutions of methyl red is monitored by following the absorbance at 520 nm and 425 nm to evaluate the performance of the IR-activated photoelectric system of the present invention.

In another demonstration of IR-activated photoelectric catalysis, the photocatalytic degradation of aqueous solutions of methylene blue dye is tested. In contrast to methyl red dyes which serve as pH indicators, methylene blue dyes are used as a redox indicator. In an oxidizing environment, solutions of methylene blue are blue, while it turns colorless upon exposure to reducing agents. Low concentrations (<20 ppm) of methylene blue have previously been shown to be decolorized and degraded by UV-irradiated TiO₂ photocatalytic systems at room temperature. In this example, methylene blue is used as a probe for the redox activity of the IR-activated photoelectric systems of the present invention.

### IR-to-UV Upconverting Phosphor Synthesis

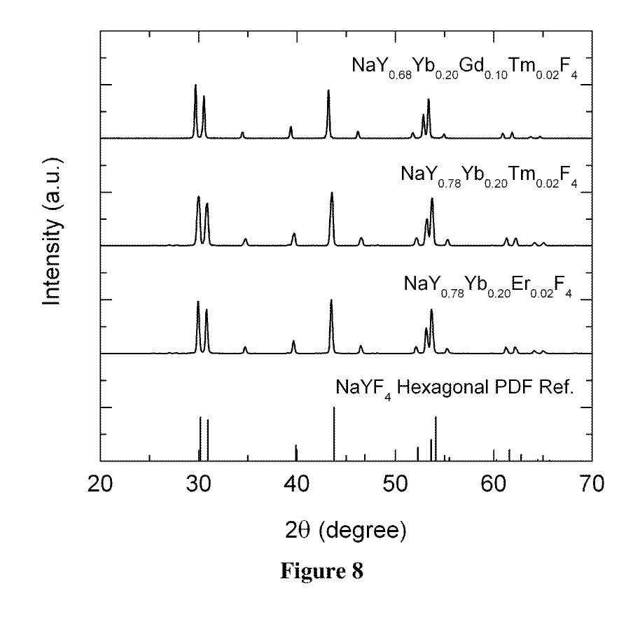

Hexagonal NaY_{0.78}Yb_{0.20}Er_{0.02}F₄, NaY_{0.78}Yb_{0.20}Tm_{0.02}F₄ and NaY_{0.68}Yb_{0.20}Gd_{0.10}Tm_{0.02} phosphors were synthesized using known solvothermal methods as shown by the XRD patterns in FIG. 8. Stoichiometric amounts of rare earth nitrates (Sigma Aldrich, St. Louis, Mo.) were mixed with 1.5 times excess sodium fluoride in ˜70 mL of water:ethanol mixture (80:20 v/v) and 8 g of PVP for 30 min. This mixture was next transferred to a 125 mL Teflon liner and heated to ˜240° C. for 4 h in a Parr pressure vessel (Parr Instrument Company, Moline, Ill.). The as-synthesized nanoparticles were washed three times in deionized water by centrifuging (Beckman-Coulter Avanti J-26 XP, Fullerton, Calif.) and dried at 70° C. in air in a mechanical convection oven (Thermo Scientific Thermolyne, Waltham, Mass.) for further powder characterization.

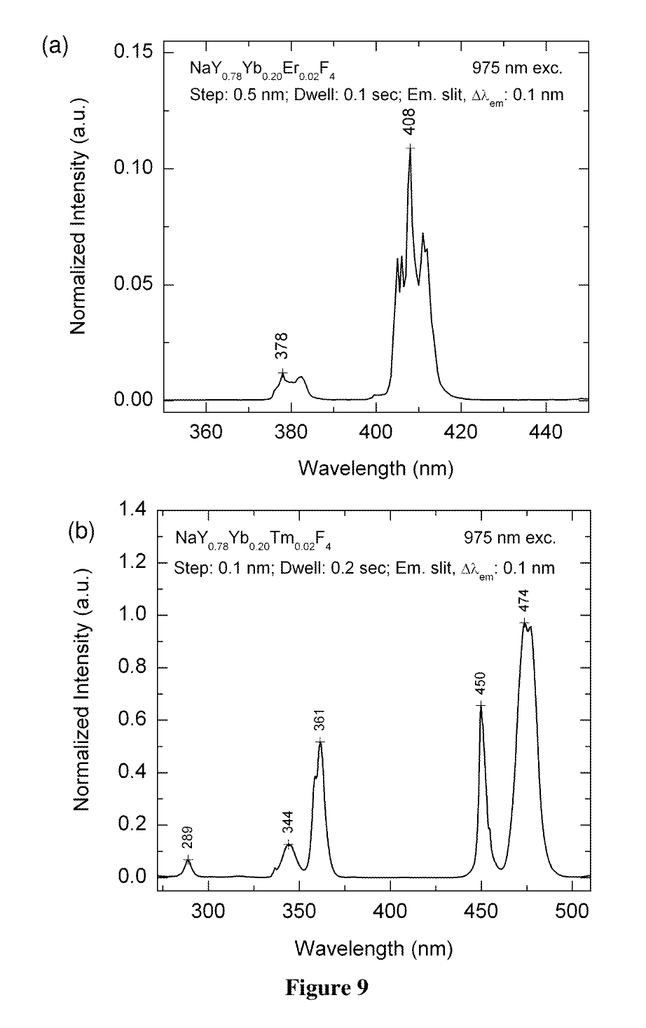

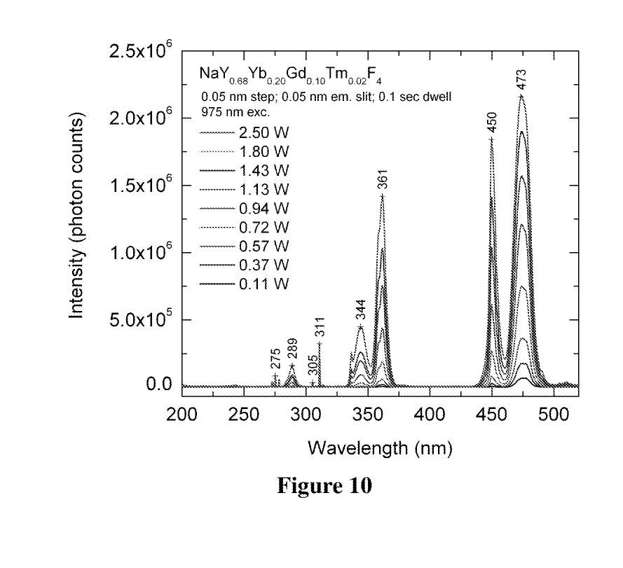

Upon excitation at 975 nm, UV emissions were observed in all phosphor samples as shown in FIGS. 9 and 10. The 378 and 408 nm emissions of NaY_{0.78}Yb_{0.20}Er_{0.02}F₄ were attributed to the ⁴G_{11/2}→⁴I_{15/2} and ²H_{9/2}→⁴I_{15/2} transitions of Er³⁺, respectively. The 289, 344, 361, 450 and 474 nm emissions of NaY₀₇₈Yb_{0.20}Tm_{0.02}F₄ were attributed to the ¹I₆→³H₆, ¹I₆→³F₄, ¹D₂→³H₆, ¹D₂→³F₄ and ¹G₄→³H₆ transitions of Tm³⁺. In addition to the peaks observed in NaY_{0.78}Yb_{0.20}Tm_{0.02}F₄, sharp peaks at 305, 311 nm and in the range of 270-281 nm were observed for NaY_{0.68}Yb_{0.20}Gd_{0.10}Tm_{0.02}, as shown in FIG. 10. The 305 and 311 nm emission peaks were attributed to the ⁶P_{5/2}→⁸S_{7/2} and the ⁶P_{7/2}→⁸S_{7/2} transitions of Gd³⁺, respectively. The emission peaks in the range of 270-281 nm were from the ⁶I_{J}→⁸S_{7/2} transitions of Gd³⁺, where J=7/2, 9/2, 11/2 and 13/2.

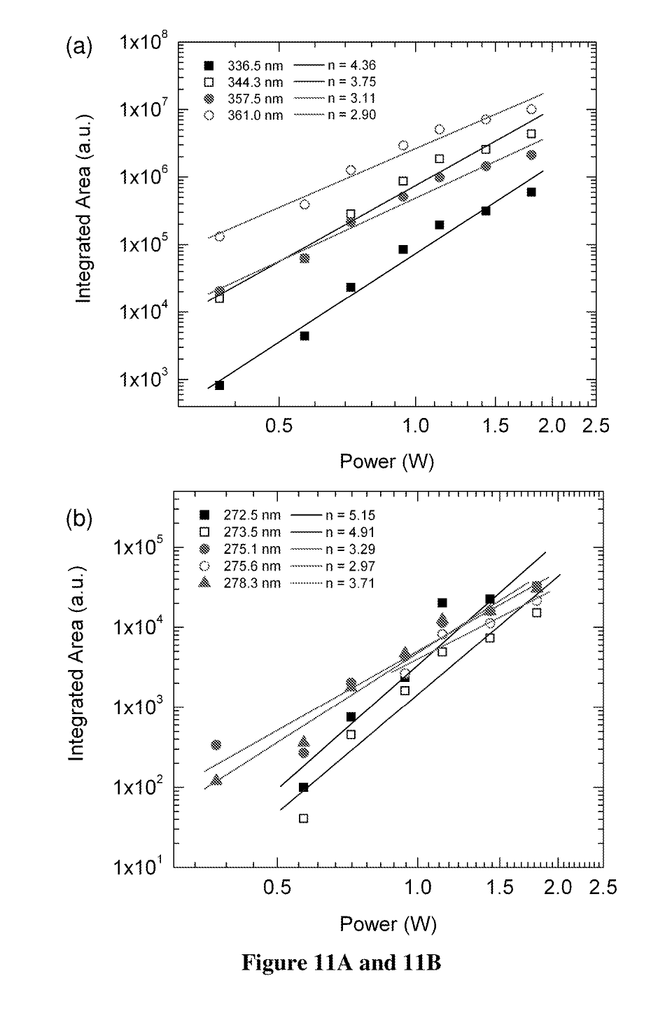

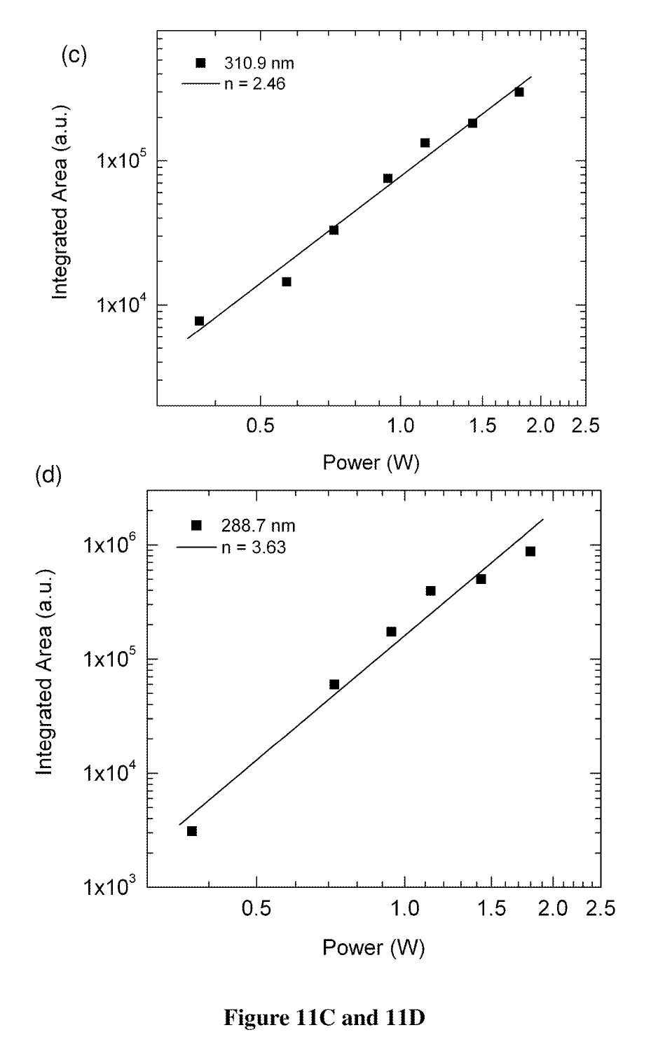

For an unsaturated upconversion process, the number of photons necessary to populate the upper emitting state can be obtained by the following relation: I_{f}∝Pⁿ, where I_{f} is fluorescence intensity, P is IR laser pumping power, and n is the number of photons required for IR-to-UV upconversion. FIG. 11 shows the log-log plots of the emission intensity as a function of excitation power for the different UV emissions of NaY_{0.68}Yb_{0.20}Gd_{0.10}Tm_{0.02}. Fluorescence intensity for each spectral peak is represented by the integrated area of emission spectra. The correspond-ding slopes (n) obtained after fitting to a linear equation are listed in Table 4. From the n values shown in Table 4 it was established that the observed UV emissions of NaY_{0.68}Yb_{0.20}Gd_{0.10}Tm_{0.02} were from upconversion processes involving 3 to 5 photons depending on the wavelength.

TABLE 4
List of n values representing number of photons required
for upconversion obtained from linear fit of double logarithmic
plots. n values rounded to nearest integer.

| Emission Peak Wavelength (nm) | n |
|---|---|
| 272.5 | 5 |
| 273.5 | 5 |
| 275.1 | 3 |
| 275.6 | 3 |
| 278.3 | 4 |
| 289 | 4 |
| 311 | 3 |
| 336.5 | 4 |
| 344.3 | 4 |
| 357.5 | 3 |
| 361.0 | 3 |

### Functional Testing of an IR-Activated Photoelectric System

An IR-to-UV upconverting rare earth doped phosphor (NaY_{0.73}Yb_{0.20}Gd_{0.05}Tm_{0.02}) and semiconductor (Degussa P25 TiO₂, Degussa AG, Dusseldorf, Germany) were coated on a glass slide. 0.2 g of as-synthesized IR-to-UV up-converting phosphors were mixed together with 0.2 g Degussa P25 TiO₂ in a solution of 0.72 mL of water, 34 mL of isopropanol (Sigma Aldrich) and 6 mL of titanium isopropoxide (Sigma Aldrich). The mixture was stirred for ˜15 h before it was next deposited on glass substrates by dip coating. Next, the solvent was allowed to vaporize under ambient conditions for 15 h.

Methyl red and methylene blue dyes were purchased from Sigma Aldrich. An aqueous solution of methyl red was prepared by dissolving 0.01 g of methyl red powder in 100 mL of deionized water. The solution (3.71×10⁻⁴ M) was next filtered using a 0.22 micron PTFE membrane (Sigma Aldrich) to remove any undissolved methyl red dye. A stock solution of methylene blue was prepared by dissolving 0.01 g of methylene blue powder in 100 mL of deionized water (2.67×10⁻⁴ M). A dilute solution (4.46×10⁻⁵ M) of 5 mL of methylene blue stock solution in 25 mL of deionized water was used for testing the IR-activated photocatalyst systems.

The glass slides were placed vertically in tubes containing the methyl red or methylene blue aqueous solutions. A near IR photodiode (BW976 BW Tek Newark, N.J.) emitting at 975 nm was used to activate photocatalytic reactions. (FIG. 14.) Circular areas with diameters of ˜1 cm on the glass slide were illuminated by the photodiode operating at ˜2 W. Testing the photocatalytic activity of the photoelectric system was completed in a dark room to eliminate any potential effects from stray light. The absorption spectra of samples taken at different time intervals were collected to evaluate the photocatalytic activity of the IR-activated photoelectric system.

### Photocatalytic Degradation of Aqueous Solutions of Methyl Red

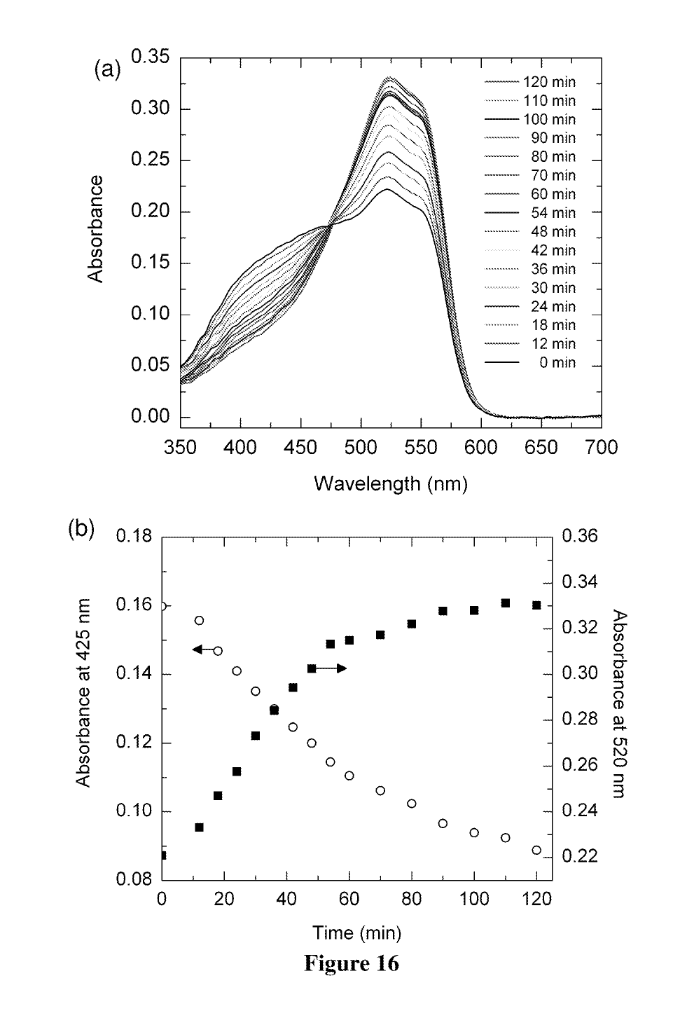

After ˜30 min of IR activation of the IR-to-UV upconverting rare earth doped phosphor and TiO₂ coating on the glass slide, gas bubbles were observed to form initially on the glass slide's surface. (See FIG. 15.) The gases eventually escaped from the solution surface and condensation was found on the tube surface. No significant change in solution temperature was observed during the gas evolution. With continued IR illumination the methyl red solution became redder, which can be attributed to decreasing solution pH. This was consistent with the absorption spectra in FIG. 16, where absorbance at 520 nm increased (increased HMR concentration) and absorbance at 425 nm decreased (decreased MR anion concentration) with time. Because the pH was expected to decrease during photocatalytic dye degradation (Table 3), observation of the solution becoming more acidic was evidence for photocatalytic activity after IR activation. It was observed from FIG. 16(b) that after ˜2 h, the absorbance at 425 and 520 nm began to approach constant values (pH˜4-5). Because only methyl red dyes adsorbed on the semiconductor surface was degraded, the observed plateau indicated that there was no methyl red on the TiO₂ surfaces. One of the possible reasons to explain the absence of methyl red on TiO₂ surface is slow dye adsorption due to the absence of agitation and/or mixing in the setup, which created a depletion zone above the photoelectric material surface.

### Photocatalytic Degradation of Aqueous Solutions of Methylene Blue

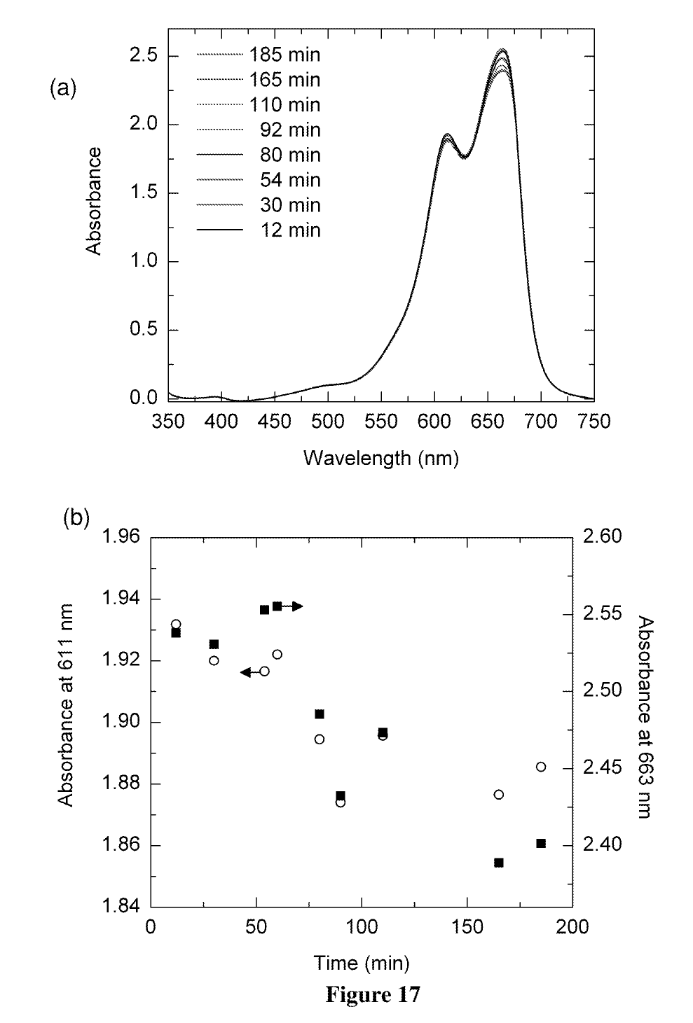

After ˜60 min of IR activation of the IR-to-UV upconverting rare earth doped phosphor and TiO₂ coating on the glass slide, gas bubbles were observed to form initially on the glass slide's surface. The gases eventually escaped from the solution surface and condensation was found on the tube surface. No significant change in solution temperature was observed during the gas evolution. Methylene blue solutions exhibit absorbance peaks at 611 and 663 nm (FIG. 17(a)). With continued IR illumination, absorbance at 611 and 663 nm were observed to decrease as shown in FIG. 17(b). The reduction in absorbance indicating methylene blue dye degradation was evidence for photocatalytic activity after IR activation. The large fluctuation in absorbance was attributed to the air-sensitive nature of methylene blue. Upon exposure to an oxidizing environment (e.g. O₂ in air or O₂ from photocatalytic oxidation of water), non-degraded methylene blue in solution can react to increase absorbance at 611 and 663 nm to give a bluer solution.

While the invention has been disclosed in connection with the preferred embodiments and methods of use, it is to be understood that many alternatives, modifications, and variations thereof are possible without departing from the present invention. Thus, the present invention is intended to embrace all such alternatives, modifications, and variations as may be apparent to those skilled in the art and encompassed within the hereinafter appended claims.

1. A photoelectric system comprising a semiconductor and a phosphorescent compound with an emission spectrum comprising photons with energy levels equal to or greater than the activation energy of said semiconductor, wherein said phosphorescent compound is characterized by said emission spectrum being produced by excitation of said phosphorescent compound with lower energy photons and the separation distance between said semiconductor and said phosphorescent compound is less than the distance at or above which scattering losses predominate.

2. The photoelectric system of claim 1, wherein said semiconductor and phosphorescent compounds are configured:
(i) so that upon excitation, said phosphorescent compound emits photons with wavelengths that create electron-hole pairs in said semiconductor that react with any water, water vapor, oxygen, carbon dioxide or organic materials in contact with said semiconductor to generate free radicals and other reactive species; or
(ii) for the photo-generation of an electric current.

3. (canceled)

4. The photoelectric system of claim 1, wherein said lower energy photons comprise photons with an energy level of about 2.0 eV or less.

5. The photoelectric system of claim 1, wherein said phosphorescent compound is excited by IR wavelength photons.

6. The photoelectric system of claim 1, wherein said phosphorescent compound is an upconverting phosphor comprising a host compound doped with one or more rare earth elements.

7. The photoelectric system of claim 6, wherein said host compound is a halide selected from the group consisting of NaYF₄, YF₃ and LaF₃.

8. The photoelectric system of claim 6, wherein said one or more rare earth elements are selected from the group consisting of ytterbium (Yb), thulium (Tm), erbium (Er) and gadolinium (Gd).

9. (canceled)

10. The photoelectric system of claim 1, wherein said upconverting phosphor is selected from the group consisting of NaYF₄:Yb—Tm, NaGdF₄:Yb—Tm LaF₃:Yb—Tm, YF₃:Yb—Tm, GdF₃:Yb—Tm, YF₃:Yb—Gd—Tm and NaYF₄:Yb—Er.

11. The photoelectric system of claim 1, wherein said semiconductor is selected from the group consisting of anatase TiO₂, rutile TiO₂, CeO₂, ZnO, Fe₂O₃, WO₃, Ta₂O₅, VO₂, ternary and quaternary metal oxides, metal sulfides, nitrides, oxynitrides, oxysulfides and mixtures thereof.

12. (canceled)

13. (canceled)

14. The photoelectric system of claim 1, wherein said semiconductor comprises a plurality of semiconductor compounds.

15-17. (canceled)

18. The photoelectric system of claim 1, comprising a mixture of semiconductor and phosphorescent compound particles having similar aspect ratios in either an ordered or disorder-ed arrangement: or
semiconductor and phosphorescent compound particles dispersed in a liquid or gas matrix or supported on a porous or non-porous solid matrix; or
semiconductor and phosphorescent compound particles, wherein the semiconductor morphologies are different from the phosphorescent compound particle morphologies; or
a phosphorescent compound embedded within a continuous matrix of a semiconductor; or
a semiconductor shell layer covering a phosphorescent compound core.

19-22. (canceled)

23. The photoelectric system of claim 1, comprising a mixture of semiconductor and phosphorescent particles having a fibrous or tubular morphology wherein said particles are arranged in an ordered configuration; or
an interpenetrating fiber network of phosphorescent compound fibers and semiconductor fibers; or
a continuous bi-layer of said semiconductor is coated onto a film or sheet of said phosphorescent compound.

24. The photoelectric system of claim 18, wherein the semiconductor and phosphorescent compound comprise particles characterized by morphologies independently selected from the group consisting of cubes, rectangular solids, cuboids, prisms, discs, pyramids, polyhedrons, multi-faceted particles, cylinders, spheres, cones, rings, tubes, acicular, angular, bent, channeled, concave, crescent, columnar, dendritic, equant, euhedral, fibrous, flaked fractal glass-like, grape-like, granular, irregular, layered, long-thin, lumpy, lath, modular, needle, oblong, plate, platelet, potato, ribbon, rippled, rod, rounded, shard, sheet, smoothed, eraser, burrito, Africa, jelly fish, worm, subhedral, striated, subangular, subsphere and twisted.

25-31. (canceled)

32. The photoelectric system of claim 1, characterized by a cellular or monolithic macrostructure.

33. The photoelectric system of claim 32, wherein said macrostructure is a foam macrostructure; or a honeycomb macrostructure; or a corrugated macrostructure; or a macrostructure comprising interconnected rods; or a macrostructure comprising interconnected fibers defining a ceramic fiber mat; or a low density closed cell structure.

34-38. (canceled)

39. A method for remediating chemical waste comprising contacting material containing organic species for remediation with the photoelectric system of claim 1 and irradiating said semiconductor system with photons of sufficient energy to excite the phosphorescent compound to emit photons of sufficient energy to activate the semiconductor to generate species that degrade or decompose said organic species.

40. (canceled)

41. (canceled)

42. A method for cleaning and sterilizing surfaces comprising irradiating a surface coated with or formed from the photoelectric system of claim 1 with photons of sufficient energy to excite the phosphorescent compound to emit photons of sufficient energy to activate the semi-conductor to generate species that kill microbes or degrade or decompose organic substances on said coated surface.

43. The method of claim 42, wherein said phosphorescent compound is a rare earth doped upconverting phosphor that upon excitation with IR wavelength photons emits photons of sufficient energy to activate said semiconductor, and said surface is selected from the group consisting of: a surface of an implantable medical device, and an outside exterior surface.

44. (canceled)

45. The method of claim 39, wherein the source of photons for exciting said phosphorescent compound is selected from the group consisting of: the sun, IR illuminators, lamps, and photodiodes.

46. (canceled)

47. (canceled)

48. A method for generating ozone comprising contacting the photoelectric system of claim 1 with an oxygen source and irradiating the photoelectric system with photons of sufficient energy to excite the phosphorescent compound to emit photons of sufficient energy to activate the semiconductor and generate species that produce ozone from oxygen.

49. A method for purifying air or water contaminated with microbes or undesirable organic compounds or organic matter comprising contacting the photoelectric system of claim 1 with an air or water source contaminated with microbes or undesirable organic compounds or organic matter and irradiating said photoelectric system with photons of sufficient energy to excite said phosphorescent compound to emit photons of sufficient energy to activate said semi-conductor and generate species that purify said air or water by killing said microbes or degrade or decompose said undesirable organic compounds or organic matter.

50. A method for producing hydrogen or a hydrocarbon fuel comprising contacting the photoelectric system of claim 1 with a source of hydrogen or a source of hydrocarbon fuel and irradiating the system with photons of sufficient energy to excite the phosphorescent compound to emit photons of sufficient energy to activate the semiconductor and generate species that decompose the hydrogen source to produce hydrogen or the hydrocarbon fuel source to produce hydrocarbon fuel.

51. The method of claim 50, wherein said hydrogen source is water or methanol.

52. The method of claim 50, wherein the hydrocarbon fuel source is biomass or carbon dioxide and the hydrocarbon fuel is methane, methanol or formaldehyde.

53-57. (canceled)

58. An architectural product, ship hull or other maritime surface coated, building facade or roof, automotive product, article of furniture, computer hardware or display or appliance surface coated with the photoelectric system of claim 1.

59. (canceled)

60. (canceled)

61. A dye-sensitized solar cell characterized by a titanium dioxide layer comprising the photoelectric system of claim 1, wherein the semiconductor is titanium dioxide.
